(19) United States
(12) Patent Application Publication
(10) Pub. No.: US 2017/0224745 A1
(43) Pub. Date: Aug. 10, 2017

(54) BACILLUS AMYLOLIQUEFACIENS PROBIOTIC COMPOSITIONS, METHODS OF PRODUCTION, AND METHODS OF USE

(71) Applicant: THE UNIVERSITY OF QUEENSLAND, St Lucia (AU)

(72) Inventor: Peter DART, St Lucia (AU)

(21) Appl. No.: 15/329,247

(22) PCT Filed: Jul. 27, 2015
(86) PCT No.: PCT/AU2015/050421

§ 371 Date: Jan. 25, 2017

(30) Foreign Application Priority Data
Jul. 25, 2014 (AU) 2014902893

(51) Int. Cl. A61K 35/742; C12N 1/02; A23K 50/10; A23K 10/18; A23K 10/30; A23K 40/10; C12N 1/20; C12N 3/00

(52) U.S. Cl. CPC A61K 35/742; C12N 1/20; C12N 1/02; C12N 3/00; A23K 10/18; A23K 10/30; A23K 40/10; A23K 50/10; A61K 2035/115

(57) ABSTRACT

A probiotic composition is provided comprising Bacillus amyloliquefaciens strain H57 bacteria that confers health and/or nutritional benefits, including methods of producing and using such a composition. Also provided is a method of treating a 5 probiotic-responsive disease, disorder and condition in an animal and a method of modulating the gastrointestinal flora of an animal, both including administering to said animal a probiotic composition comprising Bacillus amyloliquefaciens strain H57 bacteria.

### TECHNICAL FIELD

THIS INVENTION relates to probiotic compositions for animals. More particularly, this invention relates to probiotic compositions comprising Bacillus amyloliquefaciens H57 strain bacteria, methods useful for producing such compositions and methods of use.

### BACKGROUND

Probiotic supplements as single or mixed strain cultures of live microorganisms typically benefit the host by improving the properties of the indigenous microflora (Havenaar et al., 1992). The resurgence of interest in probiotics in production animal nutrition is in part because they may be an alternative to the use of antibiotics in ruminant and monogastric feeds to improve animal productivity (Nagaraja, 2012). In this regard, animal nutritionists have searched for alternative ways to replace additives, such as hormone growth promotants and antibiotics, in animal production because of public concern regarding the safety of these additives. Probiotics as live microorganisms may be a suitable alternative which could be used for the growth promotion of livestock.

Probiotics used in animal nutrition are broadly divided into bacteria and fungi (Nagaraja, 2012). Common bacterial probiotics include Lactobacillus, Bifidobacterium, Enterococcus, Streptococcus, Bacillus and Propionibacterium species (Seo et al., 2010). Probiotics have been shown to improve live weight and feed intake in monogastric animals (Alexopoulos et al., 2001; Otutumi et al., 2012), but have not been investigated to the same extent in ruminants.

Accordingly, there remains a need for a probiotic composition that facilitates an improvement in the digestion and/or utilisation of feed in ruminant and/or monogastric animals.

### SUMMARY

The present invention is predicated in part on the surprising discovery that administration of a composition comprising Bacillus amyloliquefaciens H57 strain bacteria, to monogastric and/or ruminant animals may result in improved feed conversion efficiency, dietary intake, nitrogen retention and/or weight gain in these animals.

In a first aspect, the invention provides a probiotic composition comprising a microbial culture of Bacillus amyloliquefaciens strain H57 bacteria and an acceptable carrier, diluent or excipient.

In particular embodiments, the probiotic composition further comprises a probiotic microorganism of one or more genera selected from the group consisting of Lactobacillus, Bifidobacterium, Enterococcus, Streptococcus, Bacillus, Propionibacterium, Enterococcus, Streptococcus, Pediococcus, Clostridium, Aspergillus, Candida, Saccharomyces, Megasphaera and any combination thereof.

In one embodiment, the microbial culture comprises, consists or consists essentially of spores of Bacillus amyloliquefaciens strain H57 bacteria.

In a certain embodiment, the microbial culture is lyophilised and/or freeze dried.

In one embodiment, the probiotic composition is formulated as an animal feed composition, wherein the animal feed composition comprises a pelleted, granular and/or particulate feed material. Suitably, the feed material is selected from the group consisting of palm kernel meal, wheat, sorghum, corn, soybean meal, and any combination thereof.

In another embodiment, the probiotic composition is formulated as an animal feed composition, wherein the animal feed composition is or comprises a lick block.

In one embodiment, the microbial culture is present at a concentration of about 1×10⁶ to about 1×10¹⁰ colony forming units (CFU) per gram of the animal feed composition.

In one embodiment, the microbial culture is present at a concentration so as to provide a dose of about 1×10⁷ to about 1×10¹¹ CFU per day to an animal being fed the probiotic composition.

In a particular embodiment, the animal feed composition is substantially free of antibiotics and/or antimicrobial agents.

In one embodiment, the animal feed composition is steam pelleted.

In a second aspect, the invention provides a method of preventing and/or treating a disease, disorder or condition in an animal, wherein said disease, disorder or condition is responsive to a probiotic, including the step of administering to said animal a therapeutically effective amount of a probiotic composition comprising a microbial culture of Bacillus amyloliquefaciens strain H57 bacteria to thereby prevent and/or treat the disease, disorder or condition.

In particular embodiments, the disease, disorder or condition is or results in gastrointestinal disorders, poor, delayed or stunted growth and/or reduced fecundity.

In some embodiments, these may include reduced feed conversion efficiency, reduced dietary intake, reduced weight gain, reduced egg production and/or reduced egg quality, although without limitation thereto.

In a particular embodiment, the disease, disorder or condition is diarrhoea, such as in cattle (e.g calves) or other ruminants.

In a third aspect, the invention provides a method for improving or increasing one or more properties of a monogastric animal including the step of administering a probiotic composition comprising Bacillus amyloliquefaciens strain H57 bacteria to the monogastric animal in an amount effective to facilitate improving or increasing the one or more properties of the monogastric animal.

Generally, the one or more properties of the monogastric animal may include those that relate to animal husbandry and/or food production such as animal growth and/or fecundity.

In some embodiments, the one or more properties include feed conversion efficiency, dietary intake, weight gain, egg production and/or egg quality, although without limitation thereto.

In particular embodiments of the aforementioned aspects, the Bacillus amyloliquefaciens strain H57 bacteria once administered colonizes, at least temporarily, at least a portion of a gastroinstestinal tract of the animal.

In particular embodiments of the aforementioned aspects, administration of the probiotic composition modulates one or more species or genera of microbial flora in at least a portion of a gastrointestinal tract of the animal.

In particular embodiments of the aforementioned aspects, the probiotic composition is administered by mixing the probiotic composition with a feed material and/or spraying the probiotic composition onto a feed material prior to feeding.

In another embodiment of the second and third aspects, the composition is administered by adding the composition to the animal's drinking water prior to feeding.

In a fourth aspect, the invention provides a method for modulating microbial flora in at least a portion of a gastrointestinal tract of an animal including the step of administering a probiotic composition comprising Bacillus amyloliquefaciens strain H57 bacteria to the animal in an amount effective to accomplish said modulation.

Suitably, the microbial flora include one or more bacteria of a genus selected from the group consisting of Acidaminococcus, Akkermansia, Anaerovibrio, Arthromitus, Bacteroides, Blautia, Butyrivibrio, Faecalibacterium, Coprococcus, Lachnobacterium, Lachnospira, Lactobacillus, Megasphaera, Methanobrevibacter, Mitsuokella, Prevotella, Pseudoramibacter, Roseburia, Ruminobacter, Ruminococcus, Selenomonas, Shuttleworthia, Sphaerochaeta, Staphylococcus, Streptococcus, Succiniclasticum, Succinivibrio, Turicibacter and any combination thereof.

In one embodiment, the microbial flora include one or more bacteria selected from the group consisting of Prevotella ruminicola, Prevotella copri, Roseburia faecis, Selenomonas ruminantium and any combination thereof.

In particular embodiments of the second, third and fourth aspects, the animal or monogastric animal is a non-human animal. In alternative embodiments of the second, third and fourth aspects, the animal or monogastric animal is a human.

In a fifth aspect, the invention provides a method for manufacturing a probiotic composition including the steps:

(i) growing a microbial culture of Bacillus amyloliquefaciens strain H57 bacteria in a suitable media;

(ii) substantially isolating the microbial culture from the media;

(iii) inducing sporulation of the microbial culture before and/or after step (ii); and

(iv) combining spores of Bacillus amyloliquefaciens strain H57 bacteria with an acceptable carrier.

In one embodiment, the method further includes the step of lyophilising and/or freeze drying the spores after steps (iii) and/or (iv).

In a sixth aspect, the invention provides a probiotic composition made according to the method of the fifth aspect.

Suitably, the probiotic composition of the second, third and fourth aspects is that of the first and/or fifth aspect.

Throughout this specification, unless otherwise indicated, “comprise”, “comprises” and “comprising” are used inclusively rather than exclusively, so that a stated integer or group of integers may include one or more other non-stated integers or groups of integers. Conversely, the terms “consist”, “consists” and “consisting” are used exclusively, such that a stated integer or group of integers are required or mandatory, and no other integers may be present.

The phrase “consisting essentially of” indicates that a stated integer or group of integers are required or mandatory, but that other elements that do not interfere with or contribute to the activity or action of the stated integer or group of integers are optional.

It will also be appreciated that the indefinite articles “a” and “an” are not to be read as singular indefinite articles or as otherwise excluding more than one or more than a single subject to which the indefinite article refers. For example, “a” animal includes one animal, one or more animals or a plurality of animals.

### BRIEF DESCRIPTION OF THE FIGURES

FIG. 1. Change in Lactobacillus population (normalized abundances) in the ileum due to feeding H57 to poultry. 1-12=control birds, 13-24=H57 treated birds.

FIG. 2. Change in Streptococcus population (normalized abundances) in ileum due to feeding H57 to poultry. 1-12=control birds, 13-24=H57 treated birds.

FIG. 3 Change in Bacteroides population (normalized abundances) in caecum due to feeding H57 to poultry. 1-12=control birds, 13-24=H57 treated birds.

FIG. 4. Change in Faecalibacterium population (normalized abundances) in caecum due to feeding H57 to poultry. 1-12=control birds, 13-24=H57 treated birds.

FIG. 5. Effect of supplement of B. amyloliquefaciens H57 on liveweight of pregnant and lactating ewes. Solid line: Control group; dashed line: H57 group*: P<0.05; **: P<0.01; ***: P<0.001 (between treatments within weeks).

FIG. 6. Multiple alignment tree of genomes extracted from sheep rumen fluid of both control and +H57 animals. Aligned using the genome_tree_database v1.9.9.2 and visualised using ARB v6.0.2. Genomes indicated by block arrows (i.e., 3kb_bin_35 Ben_S_15122014, 1.5kb_bin_51_Ben_S_30122014 and 3kb_bin_49_Ben_S_30122014) are those genomes that have been identified as dominant organisms within their respective populations.

FIG. 7. Liveweight change (A) and liveweight gain (B) of dairy calves due to Bacillus amyloliquefaciens H57 treatment (error bars are S.E.M.).

FIG. 8. Duration (A) of diarrhea for affected calves, proportion of calves having diarrhoea and duration of diarrhoea treatment needed for the H57 and the Control calves (error bars are S.E.M).

FIG. 9. Daily intake of pellets based on measurement of supply and refusals (A) and cumulative total dry matter intake for each weekly period (B) of the Treatment calves (solid line) and the Control calves (dashed line) (error bars are S.E.M).

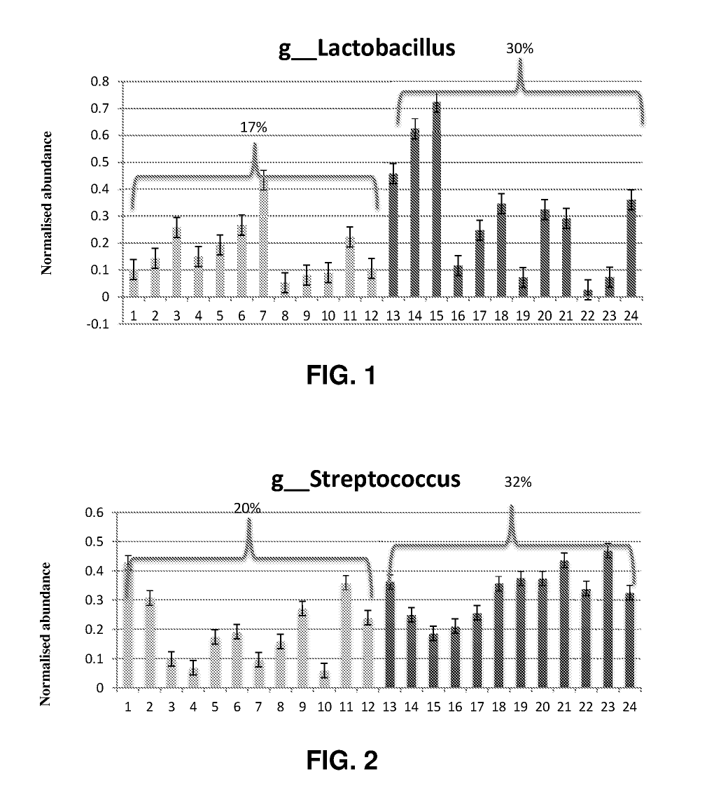

### DETAILED DESCRIPTION

The present invention arises, in part, from the discovery that feeding an animal a diet incorporating Bacillus amyloliquefaciens strain H57 bacteria may result in this bacteria colonizing the animal's gastrointestinal tract and thereby improving the microbial balance therein and providing health and/or nutritional benefits to the animal. The present invention provides a probiotic composition comprising Bacillus amyloliquefaciens strain H57 bacteria that confers health and/or nutritional benefits and methods of producing and using such a composition. Further, the present invention provides a method of modulating the gastrointestinal flora of an animal by administering a probiotic composition comprising Bacillus amyloliquefaciens strain H57 bacteria to the animal.

As generally used herein, the term “probiotic” or “probiotic microorganism” refers to one or more live microorganisms that when administered in adequate amounts to an animal may confer a health benefit to said animal. This health benefit is typically the result of the probiotic beneficially modulating the animal's gastrointestinal microbial balance or flora. Suitably, the probiotic microorganism is a bacterium or a fungus. Broadly, probiotic microorganisms may be of genera selected from the group consisting of Lactobacillus, Bifidobacterium, Enterococcus, Streptococcus, Bacillus, Propionibacterium, Enterococcus, Streptococcus, Pediococcus, Clostridium, Aspergillus, Candida, Saccharomyces and Megasphaera, although without limitation thereto.

In one aspect, the invention provides a probiotic composition comprising a microbial culture of Bacillus amyloliquefaciens strain H57 bacteria and an acceptable carrier.

Bacillus amyloliquefaciens strain H57 bacteria have been previously described in Dart, P. J. and Brown, S. M. (2005; RIRDC Reports 05/103) and have been commercially available as Hayrite™ (Biocare Australia and BASF Australia). Bacillus amyloliquefaciens strain H57 bacteria have been deposited at the National Measurement Institute, Melbourne, Australia on 27 Jul. 2015 under accession number V15/020112.

As would be appreciated by the skilled artisan, probiotic microorganisms may be autochthonous or allochthonous to the gastrointestinal tract of their animal host. Additionally, probiotic microorganisms may or may not be capable of forming spores. By way of example, lactic acid bacteria (LAB), such as Lactobacillus, Bifidobacterium or Enterococcus species, are normally autochthonous and are not capable of forming spores whereas Bacillus or Clostridium species are typically allochthonous and sporogenous.

In one embodiment, the microbial culture comprises, consists or consists essentially of spores of Bacillus amyloliquefaciens strain H57. It would be well understood that there may be issues with non-sporogenous bacteria and their use as probiotics. These may include for example, a relatively short shelf life, a narrow temperature range of the pelleting and/or formulation process and incompatibility with acidic and/or basic conditions and/or certain pharmaceutical/chemical compounds. Conversely, the spore-forming allochthonous bacteria are generally more broadly resistant to environmental conditions and/or pharmaceutical/chemical compounds and hence are typically more stable than autochthonous bacteria as probiotics in animals.

The probiotic composition comprising Bacillus amyloliquefaciens strain H57 bacteria may be in any form. Preferably, the probiotic is in a dry form, such as a powder, a lyophilisate, a spore, a suppository, a tablet, a lick block, a granulate or a capsule. In one embodiment, the microbial culture is lyophilised or freeze dried. Additionally, the probiotic Bacillus amyloliquefaciens strain H57 bacteria may be encapsulated in order to protect it from moisture. Furthermore, cells and/or spores of Bacillus amyloliquefaciens strain H57 bacteria may have undergone processing in order to increase their survival in particular conditions or environments. Accordingly, the microorganism may be coated or encapsulated, for example, in a polysaccharide, fat, starch, protein, alginate or in a sugar matrix. By way of example, the microbial culture of Bacillus amyloliquefaciens strain H57 bacteria may be in a coating, a layer, and/or a filling, or it may be admixed throughout the composition.

Non-limiting examples of acceptable carriers for the probiotic composition of the present invention include conventional carriers such as colloidal silicon dioxide, calcium silicate, magnesium silicate, magnesium trisilicate, talc, sodium aluminium silicate, potassium aluminium silicate, calcium aluminium silicate, bentonite, aluminium silicate, alginate and magnesium stearate. In a preferred embodiment, the acceptable carrier is bentonite.

The probiotic composition may further comprise one or more carriers, diluents or excipients such as thickeners, emulsifiers, pH buffers, salts, carbohydrates inclusive of sugars and sugar alcohols, lipids, water or other solvents, although without limitation thereto. With respect to carriers, the probiotic composition may comprise a pharmaceutically acceptable carrier such as fructo-oligo-saccharide (FOS) medium, or other soluble fiber, sugar, nutrient or base material for the composition, such as milk powder, with which the bacterial species can be formulated, e.g., in an orally administrable form. Other carrier media may include mannitol, inulin (a polysaccharide), polydextrose, arabinogalactan, polyolslactulose and lactitol. A wide variety of materials can be used as carrier material in the practice of the present disclosure, as will be apparent to those of ordinary skill in the art, based on the description herein. The microbial composition may be in the form of a tablet, capsule, lozenge, liquid suspension or emulsion, powder, drink, beverage or other edible or consumable form, which is of particular relevance to probiotic compositions.

The acceptable carrier, diluent or excipient may be present in an amount from about 0.015% to 20% or any range therein such as, but not limited to, about 0.03% to about 5%, or about 1% to about 15% by weight of the composition. In particular embodiments of the present invention, the acceptable carrier, diluent or excipient is present in an amount of about 0.01%, 0.02%, 0.03%, 0.04%, 0.05%, 0.06%, 0.07%, 0.08%, 0.09%, 0.1%, 0.125%, 0.15%, 0.175%, 0.2%, 0.225%, 0.25%, 0.275%, 0.3%, 0.325%, 0.35%, 0.375%, 0.4%, 0.425%, 0.45%, 0.475%, 0.5%, 0.55%, 0.6%, 0.65%, 0.7%, 0.75%, 0.8%, 0.85%, 0.9%, 0.95%, 1%, 1.5%, 2%, 2.5%, 3%, 3.5%, 4% 4.5%, 5%, 5.5%, 6%, 6.5%, 7%, 7.5%, 8%, 8.5%, 9%, 9.5%, 10%, 10.5% 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20% or any range therein, by weight of the composition. In certain embodiments of the present invention, the acceptable carrier is preferably present in an amount of about 0.015% to about 15% by weight of the composition.

In some embodiments, the probiotic composition is formulated as an animal feed composition, wherein the animal feed composition comprises a pelleted, granular and/or particulate feed material. Non-limiting examples of feed materials to be formulated with the probiotic composition include grains, such as wheat, sorghum, barley, rye, triticale and oats, vegetable protein sources, such as soybean, canola, cottonseed, sunflower, palm kernel meal, peas and lupins, and animal protein sources, such as meat meal, meat and bone meal, fish meal, poultry by-product meal, blood meal and feather meal. Suitably, the feed material is selected from the group consisting of palm kernel meal, wheat, sorghum, corn, soybean meal and any combination thereof.

In another embodiment, the animal feed composition is or comprises a lick block.

As would be appreciated by the skilled artisan, lick blocks are a practical way of supplementing major nutrients such as nitrogen, phosphorus and sulphur, particularly to ruminant animals and horses grazing either or both natural and cultivated pastures. In this regard, the block or lick in addition to Bacillus amyloliquefaciens strain H57 bacteria may contain minerals, such as zinc sulfate, copper sulfate, ferrous sulfate, manganese sulfate, cobalt chloride, potassium iodide, sodium selenite, magnesium sulfate, sodium sulfate, calcium sulfate, calcium hydrogen phosphate, sodium chloride, ammonium sulphate, dicalcium phosphate and urea, molasses, a protein meal, and/or a fibrous feed material, albeit without limitation thereto. The lick block may be made by any method known in the art, but generally, the required ingredients are mixed together and reacted with bonding, setting and/or hardening agents and pressed together into a lick block.

Suitably, the composition does not comprise hay, such as lucerne hay.

The microbial culture of Bacillus amyloliquefaciens strain H57 bacteria may be present in an amount of about 1×10⁴ to about 1×10¹¹ CFU per gram of the animal feed composition or any range therein such as, but not limited to, about 1×10⁵ to about 1×10¹⁰, or about 1×10⁶ to about 5×10⁹ CFU per gram of the animal feed composition. In particular embodiments of the present invention, the microbial culture of Bacillus amyloliquefaciens strain H57 bacteria is present in an amount of about 1×10⁴ 2×10⁴ 4×10⁴ 6×10⁴ 8×10⁴ 1×10⁵ 2×10⁵ 4×10⁵ 6×10⁵ 8×10⁵, 1×10⁶, 2×10⁶, 4×10⁶, 6×10⁶, 8×10⁶, 1×10⁷ 2×10⁷ 4×10⁷ 6×10⁷, 8×10⁷ 1×10⁸, 2×10⁸, 4×10⁸, 6×10⁸, 8×10⁸, 1×10⁹, 2×10⁹, 4×10⁹, 6×10⁹, 8×10⁹, 1×10¹⁰, 2×10¹⁰, 4×10¹⁰, 6×10¹⁰, 8×10¹⁰, 1×10¹¹ CFU per gram of the animal feed composition or any range therein. In particular preferred embodiments, the microbial culture of Bacillus amyloliquefaciens strain H57 bacteria is present in an amount of about 1×10⁶ to about 1×10¹⁰ CFU per gram of the animal feed composition.

In one embodiment, the microbial culture is present at a concentration so as to provide a dose of about 1×10⁷ to about 1×10¹¹ CFU per day to the one or more animals being fed the probiotic composition. This includes any range therein such as, but not limited to, about 1×10⁸ to about 1×10⁹, or about 5×10⁷ to about 5×10⁹ CFU per day. The dose of Bacillus amyloliquefaciens strain H57 bacteria is typically selected so as to facilitate the successful colonization, at least temporarily, of a portion of the gastrointestinal tract by the microbe and/or provide optimum health benefits to the one or more animals.

In a particular embodiment, the animal feed composition is substantially free of antibiotics and/or antimicrobial agents. In this regard, the animal feed composition is to contain little or no active antibiotics and/or antimicrobial agents, such as less than 100 ppm active antibiotic and/or antimicrobial agent.

In one embodiment, the animal feed composition is steam pelleted. In this regard, the animal feed composition may be steam pelleted by any method known in the art.

In particular embodiments, the probiotic composition further comprises one or more probiotic microorganisms of genera selected from the group consisting of Lactobacillus, Bifidobacterium, Enterococcus, Streptococcus, Bacillus, Propionibacterium, Enterococcus, Streptococcus, Pediococcus, Clostridium, Aspergillus, Candida, Saccharomyces, Megasphaera and any combination thereof.

Non-limiting examples of probiotic microorganisms that may be included in the probiotic composition of the present invention include Bifidobacterium animalis, Bifidobacterium bifidum, Bifidobacterium longum, Bifidobacterium thermophilum, Enterococcus faecalis, Enterococcus faecium, Lactobacillus acidophilus, Lactobacillus brevis, Lactobacillus bulgaricus, Lactobacillus casei, Lactobacillus cellobiosus, Lactobacillus farciminis, Lactobacillus fermentum, Lactobacillus helveticus, Lactobacillus lactis, Lactobacillus plantarum, Lactobacillus reuteri, Lactobacillus rhamnosus, Lactobacillus salivarius, Pediococcus acidilacti, Propionibacterium jensenii, Propionibacterium freudenreichii, Streptococcus thermophiles, Bacillus cereus, Bacillus licheniformis, Bacillus subtilis, Bacillus coagulans, Clostridium butyricum, Aspergillus oryzae, Candida pintolopesii, Saccharomyces cerevisiae, Saccharomyces boulardii, Megasphaera elsdenii, including and encompassing all variants, isolates and strains thereof, as are known in the art.

In particular embodiments relating to probiotics used in humans, the compositions disclosed herein may further comprise Ilactic acid bacteria (e.g. Lactobacillus species such as Lactobacillus rhamnosus, Lactobacillus casei, and Lactobacillus johnsonii) and/or Bifidobacterium although certain yeasts and other bacilli may also be used. Probiotics are commonly consumed as part of fermented foods with specially added active live cultures such as in yogurt, soy yogurt, or as dietary supplements. Although not wishing to be bound by any particular theory, probiotics are thought to beneficially affect the host by improving its intestinal microbial balance, thus inhibiting pathogens and toxin-producing bacteria. This may result in the alleviation of chronic intestinal inflammatory diseases, prevention and treatment of pathogen-induced diarrhoea, urogenital infections and atopic diseases. The probiotic microorganism(s) disclosed herein may be present at any concentration known in the art, such as from about 1×10³ to about 1×10¹⁵ CFU per gram of the probiotic composition, or any range therein including, but not limited to, about 1×10⁵ to about 1×10¹², about 1×10⁶ to about 1×10¹⁰ and about 1×10⁷ to about 1×10⁹ CFU per gram of the probiotic composition. Preferably, the concentration of the probiotic microorganism is sufficient so as to facilitate successful colonization, at least temporarily, of a portion of the gastrointestinal tract by the microbe and/or provide optimum health benefits to the one or more animals being fed the composition.

It will be understood that the composition described herein may be applicable to any animal. As used herein, the term “animal”, unless otherwise stated, includes monogastric and ruminant animals. Non-limiting examples of monogastric animals include humans, avians inclusive of poultry (e.g., chickens, ducks, geese, pigeons, quails and turkeys), pigs, horses and donkeys. Non-limiting examples of ruminant animals include cattle, sheep, goats, deer, antelope and pseudoruminants (e.g., camels, llamas and alpacas).

In a further aspect, the invention provides a method of preventing and/or treating a disease, disorder or condition in an animal, wherein said disease, disorder or condition is, at least in part, responsive to a probiotic, including the step of administering to said animal a therapeutically effective amount of a probiotic composition comprising a microbial culture of Bacillus amyloliquefaciens strain H57 bacteria to thereby prevent and/or treat the disease, disorder or condition

As used herein, “treating”, “treat” or “treatment” refers to a therapeutic intervention, course of action or protocol that at least ameliorates a symptom of the disease, disorder or condition after its symptoms have at least started to develop. As used herein, “preventing”, “prevent” or “prevention” refers to therapeutic intervention, course of action or protocol initiated prior to the onset of said disease, disorder or condition and/or a symptom of said disease, disorder or condition so as to prevent, inhibit or delay or development or progression of said disease, disorder or condition or the symptom. Further, by “responsive to a probiotic” is meant that the disease, disorder or condition is capable of and/or amenable to treatment and/or prevention by a probiotic, such as those described herein.

The terms “administering”, “administration” and the like as used herein are intended to encompass any active or passive administration of the probiotic composition to the gastrointestinal tract of an animal by a chosen route. Such routes of administration may include, for example, oral and rectal administration, but without limitation thereto. The probiotic composition may be administered by any method known in the art, including those described herein.

The term “therapeutically effective amount” describes a quantity of a probiotic composition sufficient to achieve a desired effect in the animal being treated with that probiotic composition. For example, this can be the amount of a probiotic composition comprising a microbial culture of Bacillus amyloliquefaciens strain H57 bacteria necessary to prevent and/or treat a disease, disorder or condition capable of being prevented and/or treated, at least in part, by a probiotic. In some embodiments, a “therapeutically effective amount” is sufficient to reduce or eliminate a symptom of such a disease, disorder or condition (e.g., diarrhoea). In other embodiments, a “therapeutically effective amount” is an amount sufficient to achieve a desired biological effect, for example an amount that is effective in improving or increasing feed conversion efficiency, dietary intake, weight gain, egg production and/or egg quality associated with said disease, disorder or condition.

Ideally, a therapeutically effective amount of an agent is an amount sufficient to induce the desired result without causing a substantial cytotoxic effect in the subject. The effective amount of a probiotic composition useful for reducing, alleviating and/or preventing a disease, disorder or condition will be dependent on the animal being treated, the type and severity of any associated disease, disorder and/or condition, and the manner of administration of the therapeutic composition.

In particular embodiments, the disease, disorder or condition comprises gastrointestinal disorders such as diarrhoea, reduced feed conversion efficiency, reduced dietary intake, reduced weight gain, reduced egg production and/or reduced egg quality.

As used herein, the term “diarrhoea” or “diarrhoeal disease” should be understood to mean one or a plurality of diarrhoeal subtypes, including, but not limited to, those associated with inflammatory diseases (e.g. Ulcerative colitis, Crohn's disease, Irritable Bowel Syndrome), infectious diarrhoeas (eg. caused by pathogens such as E. Coli, Salmonella, Clostridium difficile, Vibrio cholerae, Campylobacter, rotoviruses etc), drug-induced diarrhoeas (eg: chemotherapy-induced diarrhoea, antibiotic-induced diarrhoea) and allergic diarrhoeas (e.g., gluten hypersensitivity).

Thus, while the method of the invention may be employed to address a specific symptom of one or more of the above-referenced diseases, disorders or conditions, it may not necessarily treat or prevent the underlying pathology of such diseases, disorders or conditions.

As would be readily understood by the skilled artisan, the term “feed conversion efficiency” refers to a measure of an animal's efficiency in converting feed material into increases of the desired output, such as milk, meat and/or egg production. It can be calculated by dividing the total amount of feed consumed by an animal over a period of time by the gain in, for example, milk production, body weight or egg production and quality, of the animal observed over that period. Accordingly, an increased or improved feed conversion efficiency refers to a more efficient means (i.e., less feed consumption required) to achieve the desired output, such as a bringing an animal to market weight.

It would be appreciated that egg production refers to the number of eggs that a bird, for example, lays over a particular period of time. Further, egg shell quality, inclusive of external (e.g., shell) and internal (e.g., yolk and white) quality, is an important economic factor in both hatching eggs and eggs for consumption. External defects (e.g., cracks, abnormally shaped eggs, thin-shelled eggs, shell-less eggs) and internal defects (e.g., blood spots, meat spots, pale or discoloured yolks and/or whites) may lead to a decrease or downgrading of the quality of an egg. Measuring egg quality may be performed by any method known in the art.

In some embodiments, feed conversion efficiency, dietary intake, weight gain, egg production and/or egg quality are reduced or decreased if it is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the respective feed conversion efficiency, dietary intake, weight gain, egg production and/or egg quality of a control or reference sample.

In one embodiment, the animal is a non-human animal. In an alternative embodiment, the animal is a human.

In one embodiment, the Bacillus amyloliquefaciens strain H57 bacteria once administered colonizes, at least temporarily, at least a portion of a gastroinstestinal tract of the animal.

In one embodiment, administration of the composition modulates one or more species or genera of microbial flora in at least a portion of a gastrointestinal tract of the animal. As would be appreciated by the skilled artisan, microbial flora may include, but is not limited to, bacteria, protozoa, algae, fungi and/or viruses.

With respect to the colonization of Bacillus amyloliquefaciens strain H57 bacteria and/or any subsequent modulation of gastrointestinal flora, this microbe has been shown herein to produce iturin and several lipopeptides. These include surfactin, fengycin A and fengycin B. These lipopeptides and iturin may play a role, at least partly, in such colonization and/or modulation of the gastrointestinal tract and flora respectively by Bacillus amyloliquefaciens strain H57 bacteria.

As would be understood by the skilled person, the one or more microbial flora is deemed to be “modulated” when the relative or absolute number or concentration of the one or more microbial flora is increased/up regulated or decreased/down regulated when compared to a control or reference sample. By way of example, the control or reference sample may be from one or more animals known to not have been administered the probiotic composition or it may be from said animal prior to being administered the probiotic composition. The control or reference sample may be a pooled, average or an individual sample. The modulation may be temporary or permanent.

In some embodiments, the number or concentration of the one or more microbial flora is increased if it is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900% or at least about 1000% greater than the number or concentration of the one or more microbial flora in a control or reference sample.

In some embodiments, the number or concentration of the one or more microbial flora is decreased if it is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the number or concentration of the one or more microbial flora in a control or reference sample.

Accordingly, administration of the probiotic composition may result in the reappearance of one or more normally occurring microbial flora that are no longer present or are decreased in quantity from the gastrointestinal system of the animal, and/or an increase in the number or concentration to levels comparable with or higher than those typically observed in healthy animals. Furthermore, the probiotic composition may produce a decrease in the number or concentration of one or more normally occurring and/or potentially pathogenic microbial flora in the gastrointestinal system of an animal. Additionally, the probiotic composition may inhibit or prevent variations in the microbial composition and/or microbial concentrations of the gastrointestinal flora of an animal.

In a related aspect, the invention provides a method for improving or increasing feed conversion efficiency, dietary intake, weight gain, egg production and/or egg quality in a monogastric animal including the step of administering a composition comprising Bacillus amyloliquefaciens strain H57 bacteria to the monogastric animal in an amount effective to facilitate improving or increasing feed conversion efficiency, dietary intake, weight gain, egg production and/or egg quality in the monogastric animal.

Suitably, for the method of the aforementioned aspects the probiotic composition is that hereinbefore described.

In one embodiment, the monogastric animal is a non-human animal. In an alternative embodiment, the monogastric animal is a human.

In certain embodiments, feed conversion efficiency, dietary intake, weight gain, egg production and/or egg quality is improved or increased if it is more than about 0.1%, 0.5%, 1%, 2%, 3%, 4%, 5%, 10%, 20%, 30%, 40%, 50%, 60%, 70%, 80%, 90% or 100% or even more than about 150%, 200%, 250%, 300%, 450% or 500% greater than that of a control or reference sample, such as that hereinbefore described.

In one embodiment, the Bacillus amyloliquefaciens strain H57 bacteria once administered colonizes, at least temporarily, at least a portion of a gastroinstestinal tract of the monogastric animal.

In one embodiment, administration of the composition modulates one or more species or genera of microbial flora in at least a portion of a gastrointestinal tract of the monogastric animal.

In a particular embodiment, the probiotic composition is administered by mixing the probiotic composition with a feed material and/or spraying the probiotic composition onto a feed material prior to feeding. In this regard, the composition may be mixed into and/or sprayed onto the feed material by any method known in the art. Once the probiotic composition has been mixed with and/or sprayed onto the feed material, and in particular with a ground or particulate feed material, it can then, for example, be fed to the monogastric animal as a mash or dry mixture. Alternatively, the composition may be subjected to further processing that usually involves heat and/or pressure. Examples of such processing encountered in the feed industry include making lick blocks, pelleting, such as steam pelleting, roasting, steam flaking, extrusion and expansion, but without limitation thereto.

In another embodiment, the probiotic composition is administered by adding the probiotic composition to the monogastric animal's drinking water prior to feeding.

In a further aspect, the invention provides a method for modulating microbial flora in at least a portion of a gastrointestinal tract of an animal including the step of administering a probiotic composition comprising Bacillus amyloliquefaciens strain H57 bacteria to the animal in an amount effective to accomplish said modulation.

Suitably, the microbial flora include one or more bacteria of a genus selected from the group consisting of Acidaminococcus, Akkermansia, Anaerovibrio, Arthromitus, Bacteroides, Blautia, Butyrivibrio, Faecalibacterium, Coprococcus, Lachnobacterium, Lachnospira, Lactobacillus, Megasphaera, Methanobrevibacter, Mitsuokella, Prevotella, Pseudoramibacter, Roseburia, Ruminobacter, Ruminococcus, Selenomonas, Shuttleworthia, Sphaerochaeta, Staphylococcus, Streptococcus, Succiniclasticum, Succinivibrio, Turicibacter and any combination thereof.

It would be understood that this aspect includes and encompasses all species, variants, isolates and strains thereof, as are known in the art.

In one embodiment, the microbial flora include one or more bacteria selected from the group consisting of Akkermansia muciniphila, Bacteroides fragilis, Faecalibacterium prausnitzii, Lactobacillus salivarius, Prevotella ruminicola, Prevotella copri, Roseburia faecis, Selenomonas ruminantium, Streptococcus alactolyticus, and any combination thereof.

In particular embodiments, the composition is that hereinbefore described.

Suitably, the animal is either a monogastric animal or a ruminant animal, as described herein.

In one embodiment, the animal is a non-human animal. In an alternative embodiment, the animal is a human.

In yet a further aspect, the invention provides a method for manufacturing a probiotic composition including the steps:

(i) growing a microbial culture of Bacillus amyloliquefaciens strain H57 bacteria in a suitable media;

(ii) substantially isolating the microbial culture from the media;

(iii) inducing sporulation of the microbial culture before and/or after step (ii); and

(iv) combining spores of Bacillus amyloliquefaciens strain H57 bacteria with an acceptable carrier.

In one embodiment, the method further includes the step of lyophilising and/or freeze drying the spores after steps (iii) and/or (iv).

As would be appreciated by the skilled person, a suitable media for growing Bacillus amyloliquefaciens strain H57 bacteria, like most Bacillus species, may comprise a defined or relatively-simple complex media, such as that described herein. After growth of the microbial culture, Bacillus amyloliquefaciens strain H57 bacterial cells and/or spores may be substantially isolated or separated from the suitable media by any means known to those skilled in the art. Methods of isolating the microbial culture from the media may include, but are not limited to, centrifugation, vacuum filtration, membrane filtration, cell sorting or any combination thereof. In some embodiments of the present invention, water or a suitable wash solution is added to the microbial culture after isolation so as facilitate washing of the microbial culture. Thus, the isolated microbial culture may include Bacillus amyloliquefaciens strain H57 bacteria and trace amounts of water, the wash solution, the culture medium and/or by-products from the culturing process. Preferably, after isolation the microbial culture is at least 95% pure, and even more preferably, 98% to 99% pure.

With regard to step (iii) sporulation of the microbial culture may be induced by any method known in the art, such as sporulation media, including that described herein, changes in temperature (e.g., heat or cold shock), changes in pH, nutrient deprivation, sporulation-inducing agents and any combination thereof. Preferably, sporulation is induced in at least 50% of Bacillus amyloliquefaciens strain H57 bacterial cells in the microbial culture, more preferably in at least 75% of cells and even more preferably in at least 90% of cells.

In still a further aspect, the invention provides a probiotic composition produced by the method hereinbefore described.

Suitably, the probiotic composition is for use in the methods described herein.

So that the present invention may be more readily understood and put into practical effect, the skilled person is referred to the following non-limiting examples.

### Example 1

### Preparation of the Bacillus amyloliquefaciens Strain H57 Inoculum

This experiment was designed to cultivate the probiotic Bacillus amyloliquefaciens H57 in sufficient quantities for incorporation into animal feeds and induce sporulation. This was achieved by a series of fermentations in progressively larger vessels. To achieve maximum yield the culture was grown in a nutrient rich fermentation broth (Table 1) that was incubated at 29° C. for 7 hrs, sparged with air to provide oxygen to the whole vessel. This final fermentation was performed in 2×20 L steel drums with 11 L of broth culture.

At the end of the fermentation the 2 drums were used to inoculate 66 L of sporulation media (Table 2) in a 100 L fermenter. The culture was allowed to sporulate for ˜45 hrs before the contents were spun down in a Sharples high G centrifuge spinning at 15000 rpm. The harvested cells were mixed with a carrier dispersant (either sodium bentonite or skim milk powder) and water at a ratio of 1:1:3.5 (product:dispersant:water) for bentonite and 1:1:1 for milk powder. The resultant slurry was then frozen at ˜20° then freeze dried and ground to a fine powder (approximately 100 μm size particles). The freeze dried inoculum was then mixed with either more bentonite or mill run and added to the feed mix in the paddle mixer before steam pelleting. A mix ratio of approximately 1-5% was used to distribute the inoculum through the feed materials.

The results for the amount of product and bacterial counts of each run are provided in Table 3.

TABLE 1
Drum Fermenter Media. Autoclaved for 50 min at 121° C. 2 × 500 ml
broth cultures of Bacillus amyloliquefaciens H57 were used as
inoculum for each drum.

| Reagent | Quantity | |
|---|---|---|
| Glucose | 50 | g |
| Sucrose | 50 | g |
| Soytone | 50 | g |
| Yeast Extract | 20 | g |
| K₂HPO₄ | 90 | g |
| KH₂PO₄ | 30 | g |
| MgSO₄•7H₂O | 5 | g |
| CaCO₃ (precipitated) | 10 | g |
| CaCl₂ | 1 | g |
| Na₂CO₃ | 1 | g |
| FeCl₂ | 1 | g |
| Na₂SO₄ | 1 | g |
| MnSO₄ | 3 | g |
| H₁BO₃ | 0.1 | g |
| Antifoam 1520 (Agilent | 5 | ml |
| Technologies) | | |
| dH₂O | 10 | L |

TABLE 2
Sporulation media. Prepared in 100 L fermenter and sterilised for 30 min
at 125-128° C.

| Reagent | Quantity |
|---|---|
| Tap Water | 60 L |
| 5M NaOH | Until pH >10.3 |

TABLE 3
Enumeration of Bacillus amyloliquefaciens H57 inoculum

| | | Amount of | Spore count | Total Spore |
|---|---|---|---|---|
| Run | Dispersant | Product | per gram | Count |
| 3 | Sodium bentonite | 194.70 g | 7.55 × 10⁹ | 1.47 × 10¹² |
| 4 | Sodium bentonite | 176.92 g | 3.12 × 10¹⁰ | 5.52 × 10¹² |
| 5 | Sodium bentonite | 162.20 g | 2.09 × 10¹¹ | 3.39 × 10¹³ |
| 6 | Sodium bentonite | 249.57 g | 2.91 × 10¹⁰ | 7.27 × 10¹² |
| 7 | Sodium bentonite | 239.67 g | 2.40 × 10¹⁰ | 5.74 × 10¹² |
| 8 | Sodium bentonite | 314.60 g | 2.95 × 10¹⁰ | 9.27 × 10¹² |
| 9 | Sodium bentonite | 268.19 g | 3.71 × 10¹⁰ | 9.95 × 10¹² |
| 10 | Sodium bentonite | 207.48 g | 5.35 × 10¹⁰ | 1.11 × 10¹³ |
| 11 | Sodium bentonite | 334 g | 3.10 × 10¹⁰ | 1.04 × 10¹³ |
| 12 | Skim milk powder | 313.79 g | 8.70 × 10⁹ | 2.73 × 10¹² |
| Average | | 211.25 g | 4.61 × 10¹⁰ | 9.73 × 10¹² |

### Example 2

### The Effect of Probiotic H57 on Growth Performance and Nutrient Digestibility in Broilers from Day 1 to 21

Materials and Methods

Birds and Management.

One hundred and ninety five, day old broilers (Ross) were obtained from local hatchery, Woodlands Enterprises Pty Ltd, 2814 Old Gympie Rd, Beerwah Q 4519, each bird weighed and randomly allocated into each of the 7 replicate pens based on their body weight for control and 6 replicate pens (for +H57). “Pens” were cardboard boxes 95 cm×95 cm×65 cm (L×W×H). Pens were covered with wood shavings with layers of newspaper on the top for keeping the birds warm. The newspapers were changed weekly. Each pen contained a single feeding station and a water station. This resulted in 15 birds in each replicate pen placed in one of two environmental control rooms in the Queensland Animal Science Precinct (QASP), Gatton Campus, University of Queensland, one room for 6 control pens (pen 1-6) and one for 6 pens with +H57 treatment (pens 8-13) and one control pen (−H57) (pen 7) to check for cross contamination of H57 from the +H57 pens. Chicks were grown for 3 weeks. The room temperature was maintained at 31° C. on day 1, and was gradually reduced to 22° C. by 21 d of age. Feed and water were offered ad libitum.

Diets.

Both starter and grower diets are sorghum based with or without probiotic H57 to meet all the nutrient requirements (Table 4). The H57 inoculum in bentonite and all other small ingredients were added with stepwise mixing. For the inoculum this started with addition at 5% w/w to finely ground sorghum in a blender and this mix added to ground sorghum in a concrete mixer at 5%. This was then added to the concrete mixer for the final mix with the rest of the ingredients, with the final inoculum level in the feeds providing >10⁷ cells/g feed. The birds were fed with starter diet from day 1 to 14 and grower diet from 15 to 21. The H57 inoculum provided 10⁷/g feed for the starter diet in the first 14 days. During day 1-7, birds would eat c. 23 g/day thus ingesting approximately 4.6×10⁸ H57 spores/day and during day 7-14 consumption would be c. 30 g/day with slightly larger intake of H57 cells, 6.7×10⁸ cells per day. For the grower diet where birds would eat c. 100 g/bird/day the inoculum in bentonite was added to provide addition of >10⁷/g feed and each bird was estimated to intake 3×10⁹ cells of H57 per day. The amount of bentonite inoculum added to each of the feeds was about 150 g.

Measurement and Analysis.

All individual birds were weighed at days 1, 7, 14 and 21 with a one decimal flat top scale and feed intake was calculated by recording all the feed offered minus feed residue in each feeder at days 7, 14 and 21 at 8 am and the feed conversion ratio was calculated.

After weekly weighing, two birds were sampled from pens 1 to 6 (−H57 control) and pens 8 to 13 (+H57); five birds were sampled from pen 7. The birds sampled were selected as reflecting the average size for the pen. The sampled birds from pen 7 were euthanized for collecting ileal and caecal digesta samples which were then stored at −20° C. in 70 ml sample containers to be used to assess the cross contamination with H57. The two birds per pen from the rest of the treatments were euthanized for collecting gut digesta to analyse the microflora colonisation. Digesta was extruded from the GIT ileum and caecum (upper and lower ileum and caecum at the final harvest) into Eppendorf tubes and one set placed in dry ice for DNA sampling and a second set for RNA expression, frozen in liquid nitrogen and then placed in dry ice. Samples were then transported to the EcoScience Precinct, Dutton Park, and stored at −80°.

After newspapers were changed, the faecal samples were collected 8 hours later and stored at −20° C. for subsequent DNA and other component analysis. At the end of the experiment, six birds per pen were euthanized to acquire upper and lower ileal and caecal digesta for microflora and nutrient (starch and nitrogen) digestibility analyses.

### Results

The feed inoculum H57 acted very significantly as a probiotic in this trial significantly increasing body weight gain per bird over the 3 week period by 6.6% (day 7 to day 14) and 6.1% (day 14 to day 21) over uninoculated control fed birds (Tables 5 and 6). Growth was spectacular for both treated and control birds nearly doubling between day 14 and day 21 with an increase of 88%. Average daily weight gain was also significant with an increase of 7% between days 1-14 and 6.4% averaged over the whole 3 week trial period. Birds fed probiotic gained 59.4 g/day in the 15 to 21 day period, 5.4% more than control birds.

Feed intake (g/bird/day) was similar for both treatments indicating that weight gain was the result of more efficient feed conversion (Table 7). The ratio of grams of feed per unit weight gain was significantly greater for treated birds over control for days 1-14 by 8.8% and over the whole trial of 21 days by 6.8%. There was a very significant improvement in feed conversion ratio between days 8 to 14 of 12.4% (Table 8).

The European Broiler Index which takes into account mortality and daily weight gain and Feed Conversion Ratio, was also significantly increased by H57, by 17.8% initially (days 1-14) and by 15% over the whole trial (Table 9). The European Production Efficiency Factor which is based on average body weight, mortality and Feed Conversion Ratio was also significantly improved by H57 at day 7 by 7.8%, at day 14 by 17.6% and at day 21 by 14.2% (Table 10). There was no effect of H57 on starch digestibility in the GIT (Table 11).

TABLE 4
Composition of broiler starter and grower diet (percent of ingredients)

| diet | Starter | Grower |
|---|---|---|
| Sorghum | 54.72 | 59.52 |
| SBM | 32.9 | 27.8 |
| Canola meal | 3.2 | 3 |
| Meat and Bone Meal | 4.4 | 3.3 |
| Sun-soy oil | 2.94 | 4.33 |
| Lysine•HCl 78 | 0.24 | 0.22 |
| DL Methionine | 0.37 | 0.33 |
| L-Threonine | 0.1 | 0.09 |
| Limestone fine | 0.063 | 0.25 |
| MDCP Biophos | 0.114 | 0.181 |
| Salt fine | 0.23 | 0.24 |
| Sodium bicarbonate | 0.2 | 0.16 |
| Vitamin & minerals Premix* | 0.5 | 0.5 |
| Choline chloride | 0.05 | 0.06 |
| Celite | | 2 |
| Ingredient Total (%) | 100 | 100 |

*The premix was obtained from BEC which supplied per tonne of diet: Vit A: 10000000IU; Vit D₃: 2500000IU; Vit E: 30 g; Vit K₃: 2 g; Vit B₁: 1.5 g; Vit B₂: 8 g; Vit B₆: 4 g; Vit B₁₂: 20 mg; D-Calcium pantothenate: 15 g; Folic acid: 2 g; Nicotinic acid: 45 g; Biotin: 135 mg; Co: 200 mg; Cu: 6 g; Fe: 50 g; I: 750 mg; Mn: 75 g; Mo: 1 g; Se: 150 mg; Zn: 60 g

TABLE 5
Body weight (g/bird)

Body weight (g/b)

| | Day 1 | Day 7 | Day 14 | Day 21 |
|---|---|---|---|---|
| Control | 38.1 | 165.2 | 448.3^{b} | 844.7^{b} |
| H57 | 38.1 | 169.3 | 477.7^{a} | 895.9^{a} |
| SEM | 0.03 | 2.37 | 5.87 | 10.82 |
| LSD_{0.05} | 0.08 | 7.46 | 18.50 | 34.09 |
| P value | 0.570 | 0.248 | 0.05 | 0.007 |

Means within columns followed by different superscripts are significantly different at P < 0.05

TABLE 6
Average daily gain (g/bird/day)

| | Average daily gain (g/b/d) | | | | |
|---|---|---|---|---|---|
| | Day 1-7 | Day 8-14 | Day 15-21 | Day 1-14 | Day 1-21 |
| Control | 18.2 | 40.5^{b} | 56.2 | 28.4^{b} | 34.8^{b} |
| H57 | 18.8 | 44.0^{a} | 59.4 | 30.4^{a} | 37.2^{a} |
| SEM | 0.33 | 0.73 | 1.13 | 0.36 | 0.41 |
| LSD_{0.05} | 1.03 | 2.29 | 3.58 | 1.14 | 1.30 |
| P value | 0.223 | 0.007 | 0.075 | 0.004 | 0.002 |

Means within columns followed by different superscripts are significantly different at P < 0.05

TABLE 7
Feed intake (g/b/d)

| | Feed intake (g/bird/day) | | | | |
|---|---|---|---|---|---|
| | Day 1-7 | Day 8-14 | Day 15-21 | Day 1-14 | Day 1-21 |
| Control | 18.2 | 55.0 | 85.0 | 35.1 | 48.8 |
| H57 | 18.0 | 53.3 | 86.8 | 34.3 | 48.9 |
| SEM | 0.39 | 1.91 | 1.22 | 0.98 | 0.85 |
| LSD_{0.05} | 1.23 | 6.02 | 3.85 | 3.10 | 2.68 |
| P value | 0.82 | 0.541 | 0.305 | 0.565 | 0.943 |

TABLE 8
Feed conversion ratio (g feed/g gain)

| | Feed conversion ratio (g feed/g gain) | | | | |
|---|---|---|---|---|---|
| | Day 1-7 | Day 8-14 | Day 15-21 | Day 1-14 | Day 1-21 |
| Control | 1.00 | 1.36^{a} | 1.51 | 1.23^{a} | 1.35^{a} |
| H57 | 0.96 | 1.21^{b} | 1.46 | 1.13^{b} | 1.27^{b} |
| SEM | 0.02 | 0.04 | 0.02 | 0.03 | 0.02 |
| LSD_{0.05} | 0.06 | 0.12 | 0.08 | 0.09 | 0.07 |
| P value | 0.151 | 0.024 | 0.163 | 0.027 | 0.022 |

Means within columns followed by different superscripts are significantly different at P < 0.05

TABLE 9
European broiler index

| | European Broiler Index (EBI) ¹ | | | | |
|---|---|---|---|---|---|
| Treatment | Day 1-7 | Day 8-14 | Day 15-21 | Day 1-14 | Day 1-21 |
| Control | 179.4^{b} | 296.2^{b} | 366.8 | 225.9^{b} | 249.0^{b} |
| H57 | 195.2^{a} | 358.1^{a} | 402.4 | 266.2^{a} | 286.5^{a} |
| SEM | 4.46 | 11.04 | 15.54 | 6.49 | 8.38 |
| LSD_{0.05} | 14.07 | 34.79 | 48.97 | 20.46 | 26.40 |
| P value | 0.032 | 0.003 | 0.136 | 0.001 | 0.010 |

¹Average daily gain (g) × liveability (%)/(10 × FCR)
Means within columns followed by different superscripts are significantly different at P < 0.05

TABLE 10
European production efficiency factor

| | European production efficiency factor (EPEF) ² | | |
|---|---|---|---|
| Treatment | Day 7 | Day 14 | Day 21 |
| Control | 233.4^{b} | 254.3^{b} | 287.7^{b} |
| H57 | 251.7^{a} | 299.0^{a} | 328.6^{a} |
| SEM | 5.16 | 6.65 | 8.66 |
| LSD_{0.05} | 16.26 | 20.97 | 27.29 |
| P value | 0.031 | 0.001 | 0.007 |

² Average body weight (kg) × liveability (%) × 100/FCR × age (day)
Means within columns followed by different superscripts are significantly different at P < 0.05

TABLE 11
Starch digestibility (%) in different sections of gastrointestinal tract

| | Starch digestibility (%) | | |
|---|---|---|---|
| Treatment | Jejunum | Upper ileum | Lower ileum |
| Control | 70.3 | 88.0 | 92.34 |
| H57 | 68.1 | 90.3 | 93.8 |
| SEM | 2.66 | 1.39 | 0.84 |
| LSD_{0.05} | 8.50 | 4.46 | 2.68 |
| P Value | 0.561 | 0.267 | 0.264 |

### Example 3

### The Effects of Probiotics Bacillus amyloliquefaciens H57 on Gastrointestinal Microbial Community in Broiler Chicken

The populations of B. amyloliquefaciens H57 in Gastro Intestinal Tract (GIT) content were quantified by real time qPCR method using a gene specific to B. amyloliquefaciens (pgsB) (Yong, Zhang et al. 2013).

### Materials and Methods

### Samples and Experimental Design

Samples for this study were collected from a broiler feeding trial performed as per Example 2 above. Two birds from each replicate were randomly selected and euthanized on day 21 and about 0.5 g samples of digesta from the ileum and caeca were collected by squeezing the digesta into 1.5 ml Eppendorf tubes. The samples were immediately frozen in liquid nitrogen and stored at −80° C.

### DNA Extraction

DNA from digesta samples was extracted by using modified repeated bead beating plus column (RBB+C) method (Kawai, Ishii et al. 2004) and the QIAamp Fast DNA Stool Mini Kit (QIAGEN, Velno, The Netherlands). Briefly, 0.2 g of digesta samples were weighed into sterile bead beating tubes containing 0.5 g of 0.1 mm zirconia beads and suspended in 1 ml of lysis buffer. The suspension was homogenized twice in a mini bead beater (BioSpec Products Inc, Oklahoma, USA) for 5 minutes each, then heated at 70° C. for 5 minutes followed by centrifugation at 20,000 g for 1 minute to separate bacterial genomic DNA from the digesta. The separated supernatant was then treated with 1 ml of InhibitEX buffer from the kit to neutralize any PCR inhibitors present in the digesta samples followed by centrifugation at 20,000 g for 6 minutes to separate DNA from any debris present in the samples and incubated at 37° C. for one hour with 20 μl (40 mg/ml) of DNase free RNase for ileal samples or 30 μl (40 mg/ml) of DNase free RNase for caecal samples. The samples were then transferred into 15 ml Falcon tubes containing 25 μl of Proteinase K, added 600 μl of buffer AL, vortex mixed and heated at 70° C. for 10 minutes. 1.3 ml of absolute ethanol was added to the sample and all the liquid in the tube spun down through a QIAamp spin column by adding 600 μl at a time. DNA in the column was washed with 500 μl of AW1 and AW2 according to the manufacturer's directions and finally eluted with either 100 μl (ileum) or 200 μl (caecum) of elution buffer. DNA concentration of the samples to be sequenced by illumina sequencing technique was measured by using Qubit fluorometer (Thermo Fisher Scientific In, Victoria, Australia). The extracted genomic DNA was stored at −20° C. until further analysis.

### Microbial Profiling in Ileum and Caeca

To prepare for the sequencing, concentration of DNA in the genomic DNA samples was measured by using Qbit. Then, 20 μl of DNA samples with 5 μg/ml concentration was prepared by diluting the samples with the required amount of sterile deionized water. Universal primer pair 926F and 1392R were chosen for the amplification of 16S rRNA gene of DNA samples to be sequenced and the amplified DNA amplicons were sequenced by the Australian Centre for Ecogenomics at the University of Queensland using Illumina sequencing technique as described below in example 4.

The 16S rRNA sequences were clustered into operational taxonomic units (OTUs) at 97% DNA sequence similarity. Any OTU having less than 0.05% abundance was rejected. OTUs were then identified by using Basic Local Alignment Search Tool (BLAST) (Altschul, Gish et al. 1990) against the reference database and an OTU table with normalized abundance for each OTU was generated.

### Results

### Populations of H57 in the GIT of Chicken

The average number of B. amyloliquefaciens H57 cells in the ileum of H57(+) birds on day 14 was 1.1×10⁷ cells/g while on day 21 there were 1.05×10⁷ cells/g of digesta.

The average number of B. amyloliquefaciens H57 in the caecum digesta of H57(+) birds on day 14 was 2.17×10⁶ cells/g and on day 21 was 1.4×10⁶ cells/g. The difference in the numbers of H57 between day 14 and day 21 was not significant. However, the differences in the population of H57 between ileum and caecum were statistically significant (Table 12).

TABLE 12
B. amyloliquefaciens H57in the ileum and caecum
of H57+ fed chickens on day 14 and day 21

| Replicates | Day | Ileum | Caecum |
|---|---|---|---|
| R1 | 14 | 1.21E+07 | 2.25E+06 |
| | 21 | 1.07E+07 | 4.57E+06 |
| R2 | 14 | 1.05E+07 | 7.61E+05 |
| | 21 | 6.64E+06 | 1.82E+06 |
| R3 | 14 | 1.17E+07 | 9.92E+05 |
| | 21 | 1.09E+07 | 1.94E+06 |
| R4 | 14 | 6.75E+06 | 1.03E+06 |
| | 21 | 1.16E+07 | 1.17E+06 |
| R5 | 14 | 1.18E+07 | 7.09E+05 |
| | 21 | 1.55E+07 | 8.42E+05 |
| R6 | 14 | 1.04E+07 | 5.03E+05 |
| | 21 | 1.03E+07 | 2.70E+06 |

The number of H57 bacteria in the control samples was below the detectable limit by the PCR technique.

Community Profiling of the GIT of Broiler Chickens with and without H57 their Feed.

The sequencing data showed that feeding B. amyloliquefaciens H57 to poultry affected the gastrointestinal microbial population, with substantial differences in microbial populations between treated and control birds.

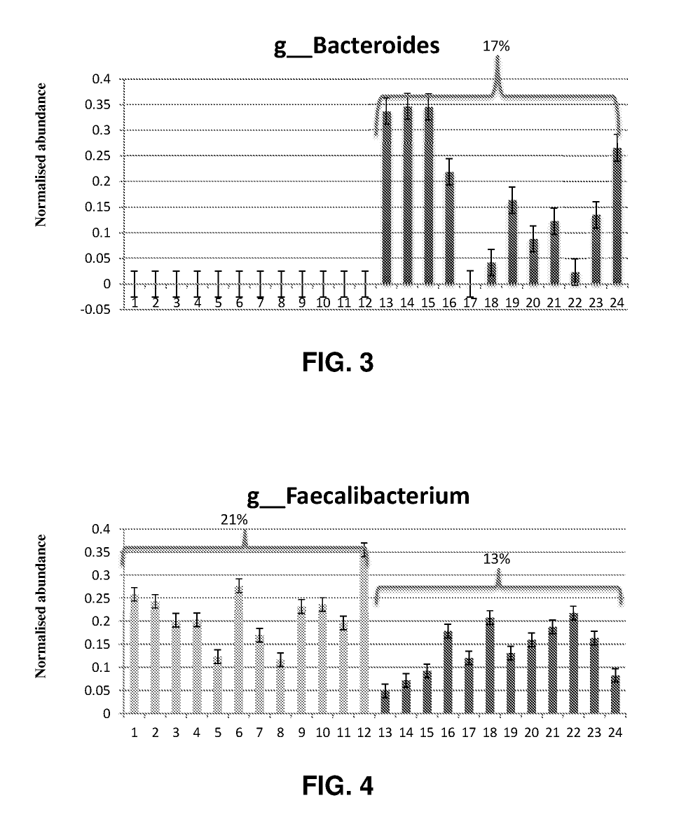

Streptococcus and Lactobacillus were the dominant genera in the ileum (Table 13). The most prominent changes in the ileum due to feeding of Bacillus amyloliquefaciens H57 was an increase in the population of Lactobacillus and Streptococcus (FIGS. 2, and 3). The population of Lactobacillus as a percentage of the total increased from 17% to 30% while that of Streptococcus increased from 20% to 32% (Table 13). Similarly, the family Enterobacteriaceae also increased. On the other hand populations of the genus Turicibacter and Staphylococcus and families Peptostreptococcaceae and Clostridiaceae decreased in the ileum (Table 13).

Similarly, Faecalibacterium is the dominant genus in the caecum of control birds not fed H57 while Bacteroides was the dominant genus in the H57 treated birds (Table 13). The most prominent change in the caecum due to feeding H57 was the dramatic increase in the population of Bacteroides. Although there were negligible Bacteroides in the caecum of control birds, Bacteroides was the most dominant genus in the H57 treated birds 17.4% of the total microbial population (Table 13, FIG. 3). In contrast, the population of Faecalibacterium decreased from 21% to 13% in the H57 treated birds (FIG. 4).

Microbial profiling showed that the composition of microbes in the ileum and caecum were significantly different (Table 13). Feeding H57 to chickens dramatically changed the microbial community structure and abundances of particular bacteria.

TABLE 13
Average relative abundances of OTUs generated from ileal and caecal digesta of broiler chicken

| | | Ileum | | Caeca | |
|---|---|---|---|---|---|
| OTU ID | Taxonomy | Control | +H57 | Control | +H57 |
| New.ReferenceOTU69 | f_{——}Turicibacteraceae; | 0.2349% | 0.0318% | 0.0002% | 0.0001% |
| | g_{——}Turicibacter; s_{——} | | | | |
| New.ReferenceOTU131 | f_{——}Lachnospiraceae; g_{——}; s_{——} | 0 | 0.0014% | 0.1679% | 0.1244% |
| New.ReferenceOTU637 | f_{——}Ruminococcaceae; g_{——}; s_{——} | 0 | 0.0007% | 0.1184% | 0.0590% |
| 177902 | o_clostridiales; f_{——}; g_{——}; s_{——} | 0 | 0.0002% | 0.1362% | 0.0607% |
| 743693 | o_clostridiales; f_{——}; g_{——}; s_{——} | 0.0002% | 0.0006% | 0.1323% | 0.0563% |
| New.ReferenceOTU466 | f_{——}Ruminococcaceae; g_{——}; s_{——} | 0.0004% | 0.0016% | 0.1392% | 0.0874% |
| 289771 | o_clostridiales; f_{——}; g_{——}; s_{——} | 0.0017% | 0.0030% | 0.2731% | 0.4970% |
| 539647 | f_{——}Lactobacillaceae; | 0.8277% | 0.9226% | 0.0039% | 0.0022% |
| | g_{——}Lactobacillus; s_{——} | | | | |
| New.ReferenceOTU728 | f_{——}Lachnospiraceae; g_{——}Blautia; s_{——} | 0.2291% | 0.0341% | 0.0001% | 0 |
| 1117319 | f_{——}Leuconostocaceae; g_{——}; s_{——} | 0.2482% | 0.1385% | 0.0076% | 0.0068% |
| 4460021 | f_{——}Ruminococcaceae; | 0.0022% | 0.0145% | 0.6404% | 0.4490% |
| | g_{——}Ruminococcus; s_{——} | | | | |
| 4388645 | f_{——}Enterococcaceae; | 2.1696% | 1.9413% | 0.0154% | 0.0126% |
| | g_{——}Enterococcus; s_{——} | | | | |
| 3438642 | f_{——}Lachnospiraceae; | 0.0128% | 0.0179% | 0.3246% | 0.1559% |
| | g_{——}[Ruminococcus]; s_{——} | | | | |
| New.ReferenceOTU232 | f_{——}Streptococcaceae; | 1.8340% | 1.3572% | 0.0036% | 0.0038% |
| | g_{——}Streptococcus; s_{——}alactolyticus | | | | |
| 1021172 | f_{——}Lactobacillaceae; | 3.0753% | 5.1212% | 0.0532% | 0.0551% |
| | g_{——}Lactobacillus; s_{——}salivarius | | | | |
| 302880 | f_{——}Streptococcaceae; | 0.7210% | 0.3952% | 0.0018% | 0.0020% |
| | g_{——}Streptococcus; s_{——} | | | | |
| 4473883 | f_{——}Streptococcaceae; | 16.6060% | 28.8212% | 0.7409% | 0.7221% |
| | g_{——}Streptococcus; s_{——}alactolyticus | | | | |
| 4357315 | f_{——}Ruminococcaceae; | 0.0003% | 0.0034% | 0.1757% | 0.2037% |
| | g_{——}Oscillospira; s_{——} | | | | |
| 157121 | o_{——}clostridiales; f_{——}; g_{——}; s_{——} | 0.0010% | 0.0011% | 0.1083% | 0.0916% |
| 169364 | f_{——}Lachnospiraceae; g_{——}; s_{——} | 0.0003% | 0.0028% | 0.2193% | 0.1343% |
| 4404361 | o_{——}clostridiales; f_{——}; g_{——}; s_{——} | 0.0000% | 0.0000% | 0.2706% | 0.0134% |
| 182643 | f_{——}Peptostreptococcaceae; g_{——}; s_{——} | 1.6723% | 0.8990% | 0.0136% | 0.0106% |
| New.ReferenceOTU180 | f_{——}Turicibacteraceae; | 0.3284% | 0.0420% | 0.0001% | 0.0002% |
| | g_{——}Turicibacter; s_{——} | | | | |
| 363997 | f_{——}Ruminococcaceae; g_{——}; s_{——} | 0.0018% | 0.0020% | 0.2747% | 0.1292% |
| 717336 | f_{——}Aerococcaceae; g_{——}Aerococcus; | 0.1801% | 0.1234% | 0.0013% | 0.0023% |
| | s_{——} | | | | |
| 158321 | o_{——}clostridiales; f_{——}; g_{——}; s_{——} | 0.0025% | 0.0131% | 0.4178% | 0.2712% |
| New.ReferenceOTU421 | f_{——}Ruminococcaceae; g_{——}; s_{——} | 0.0001% | 0.0022% | 0.1303% | 0.1491% |
| 585220 | f_{——}Erysipelotrichaceae; g_{——}cc_115; | 0.0006% | 0.0029% | 0.1247% | 0.0905% |
| | s_{——} | | | | |
| New.ReferenceOTU368 | f_staphylococcaceae; | 0.1352% | 0.1580% | 0.0001% | 0.0003% |
| | g_{——}Macrococcus; s_{——}caseolyticus | | | | |
| 157382 | f_{——}Ruminococcaceae; g_{——}; s_{——} | 0.0003% | 0.0034% | 0.1500% | 0.2974% |
| New.ReferenceOTU25 | f_{——}Peptostreptococcaceae; g_{——}; s_{——} | 0.2330% | 0.1190% | 0.0004% | 0.0004% |
| 574528 | f_{——}Clostridiaceae; g_{——}; s_{——} | 3.4897% | 0.2171% | 0.0264% | 0.0022% |
| 851733 | f_{——}Lactobacillaceae; | 0 | 0.8744% | 0 | 0.0423% |
| | g_{——}Lactobacillus; s_{——} | | | | |
| New.ReferenceOTU261 | o_{——}clostridiales; f_{——}; g_{——}; s_{——} | 0 | 0.0010% | 0.1483% | 0.0559% |
| 4306262 | f_{——}Verrucomicrobiaceae; | 0.0001% | 0.0051% | 0 | 0.8548% |
| | g_{——}Akkermansia; s_{——}muciniphila | | | | |
| New.ReferenceOTU578 | f_{——}Lachnospiraceae; g_{——}; s_{——} | 0.0006% | 0.0168% | 0.1047% | 0.0876% |
| 157516 | f_{——}Lachnospiraceae; | 0.0035% | 0.0027% | 0.4339% | 0.1935% |
| | g_{——}[Ruminococcus]; s_{——} | | | | |
| 311732 | f_{——}Ruminococcaceae; g_{——}; s_{——} | 0.0007% | 0.0072% | 0.5903% | 0.5229% |
| 732934 | f_staphylococcaceae; | 1.4666% | 0.2713% | 0.0269% | 0.0063% |
| | g_{——}Staphylococcus; s_{——} | | | | |
| 21195 | f_{——}Ruminococcaceae; g_{——}; s_{——} | 0 | 0.0013% | 0.0574% | 0.2672% |
| 211212 | f_{——}Lachnospiraceae; g_{——}; s_{——} | 0.0010% | 0.0026% | 0.0918% | 0.1154% |
| New.ReferenceOTU317 | f_{——}Ruminococcaceae; g_{——}; s_{——} | 0.0000% | 0.0009% | 0.2473% | 0.1561% |
| 201658 | f_{——}Ruminococcaceae; | 0.0019% | 0.0071% | 0.8001% | 0.3709% |
| | g_{——}Faecalibacterium; s_{——}prausnitzii | | | | |
| 4296701 | f_{——}Ruminococcaceae; | 0.0005% | 0.0037% | 0.1965% | 0.1151% |
| | g_{——}Oscillospira; s_{——} | | | | |
| 546456 | f_{——}Ruminococcaceae; g_{——}; s_{——} | 0 | 0.0067% | 0.0661% | 0.3953% |
| 4480359 | f_{——}Ruminococcaceae; g_{——}; s_{——} | 0.0003% | 0.0048% | 0.1756% | 0.1035% |
| New.ReferenceOTU292 | f_{——}Lactobacillaceae; | 0.9622% | 0.3931% | 0.0009% | 0.0007% |
| | g_{——}Lactobacillus; s_{——}salivarius | | | | |
| 16195 | f_{——}Clostridiaceae; g_{——}Candidatus | 0.5780% | 0.5160% | 0.0054% | 0.0062% |
| | Arthromitus; s_{——} | | | | |
| New.ReferenceOTU517 | f_{——}Rhodobacteraceae; g_{——}; s_{——} | 0.0122% | 0.2119% | 0.0002% | 0.0017% |
| 193125 | f_{——}Lachnospiraceae; | 0.0202% | 0.0044% | 0.3680% | 0.0893% |
| | g_{——}[Ruminococcus]; s_{——} | | | | |
| 925494 | f_staphylococcaceae; | 0.4192% | 0.0345% | 0.0034% | 0 |
| | g_{——}Staphylococcus; s_{——}aureus | | | | |
| New.ReferenceOTU234 | f_{——}Ruminococcaceae; g_{——}; s_{——} | 0.0001% | 0.0032% | 0.2014% | 0.1050% |
| 4476964 | f_{——}Bacteroidaceae; g_{——}Bacteroides; | 0.0000% | 0.0045% | 0 | 1.1849% |
| | s_{——}fragilis | | | | |
| 192720 | f_{——}Ruminococcaceae; g_{——}; s_{——} | 0.0003% | 0.0001% | 0.2327% | 0.0172% |
| 297287 | f_{——}Coriobacteriaceae; | 0.0069% | 0.0030% | 0.1949% | 0.0608% |
| | g_{——}Adlercreutzia; s_{——} | | | | |
| New.ReferenceOTU6 | f_{——}Lactobacillaceae; | 0.7863% | 0.2552% | 0.0006% | 0.0014% |
| | g_{——}Lactobacillus; s_{——} | | | | |
| 4482944 | f_{——}Streptococcaceae; | 0.9271% | 0.4157% | 0.0138% | 0.0030% |
| | g_{——}Lactococcus; s_{——} | | | | |
| 157297 | f_{——}Ruminococcaceae; | 0.0116% | 0.1690% | 6.6540% | 5.1181% |
| | g_{——}Faecalibacterium; s_{——}prausnitzii | | | | |
| 4407747 | f_{——}Ruminococcaceae; g_{——}; s_{——} | 0.0002% | 0.0012% | 0.1348% | 0.0905% |
| New.ReferenceOTU29 | f_{——}Ruminococcaceae; | 0 | 0.0007% | 0.2277% | 0.0703% |
| | g_{——}Faecalibacterium; s_{——}prausnitzii | | | | |
| 199368 | f_{——}Ruminococcaceae; | 0.0005% | 0.0007% | 0.1518% | 0.0609% |
| | g_{——}Ruminococcus; s_{——} | | | | |
| New.ReferenceOTU248 | f_{——}Ruminococcaceae; | 0.0001% | 0.0017% | 0.2106% | 0.1964% |
| | g_{——}Faecalibacterium; s_{——}prausnitzii | | | | |
| New.ReferenceOTU377 | c_{——}Mollicutes; o_{——}RF39; f_{——}; g_{——}; | 0.0006% | 0.0015% | 0.1767% | 0.1561% |
| | s_{——} | | | | |
| 194836 | f_{——}Ruminococcaceae; | 0.0016% | 0.0107% | 0.1693% | 1.0403% |
| | g_{——}Faecalibacterium; s_{——}prausnitzii | | | | |
| 4446120 | f_{——}Ruminococcaceae; | 0 | 0.0011% | 0.1317% | 0.0822% |
| | g_{——}Oscillospira; s_{——} | | | | |
| 40798 | f_{——}Ruminococcaceae; g_{——}; s_{——} | 0.0003% | 0.0162% | 0.0978% | 0.4481% |
| 291392 | f_{——}Lachnospiraceae; g_{——}Blautia; s_{——} | 0.0361% | 0.0236% | 0.1321% | 0.2597% |
| New.ReferenceOTU127 | k_{——}Archaea; p_{——}Crenarchaeota; | 0.4487% | 0.4126% | 0.0068% | 0.0038% |
| | c_{——}MCG; o_{——}; f_{——}; g_{——}; s_{——} | | | | |
| New.ReferenceOTU658 | o_{——}RF39; f_{——}; g_{——}; s_{——} | 0.0003% | 0.0040% | 0.1350% | 0.0505% |
| 582181 | f_{——}Anaeroplasmataceae; | 0 | 0.0029% | 0.2081% | 0.0782% |
| | g_{——}Anaeroplasma; s_{——} | | | | |
| 519763 | f_{——}Ruminococcaceae; | 0.0003% | 0.0045% | 0.1460% | 0.2093% |
| | g_{——}Oscillospira; s_{——} | | | | |
| 182764 | f_{——}Lactobacillaceae; | 0.1238% | 0.4462% | 0.0011% | 0.0019% |
| | g_{——}Lactobacillus; s_{——} | | | | |
| 4424737 | f_{——}Enterobacteriaceae; g_{——}; s_{——} | 0.0849% | 0.5905% | 0.1073% | 0.1429% |
| 4473358 | f_{——}Peptostreptococcaceae; g_{——}; s_{——} | 14.6287% | 6.4009% | 0.1633% | 0.1431% |
| 4433833 | f_{——}Enterobacteriaceae; g_{——}; s_{——} | 0.0133% | 0.2797% | 0.0150% | 0.0329% |
| 650615 | f_{——}Corynebacteriaceae; | 1.6667% | 1.2258% | 0.0222% | 0.0589% |
| | g_{——}Corynebacterium; s_{——}stationis | | | | |
| 1132942 | f_{——}Ruminococcaceae; g_{——}; s_{——} | 0.0007% | 0.0042% | 0.1158% | 0.2078% |
| 151870 | f_{——}Erysipelotrichaceae; | 0.0031% | 0.0047% | 0.2887% | 0.1355% |
| | g_{——}Coprobacillus; s_{——} | | | | |
| 157693 | f_{——}; g_{——}; s_{——} | 0 | 0.0005% | 0.1886% | 0.1034% |
| New.ReferenceOTU372 | f_{——}Ruminococcaceae; g_{——}; s_{——} | 0 | 0.0003% | 0.1675% | 0.0903% |
| 129401 | f_{——}Ruminococcaceae; | 0.0002% | 0.0033% | 0.4605% | 0.3033% |
| | g_{——}Oscillospira; s_{——} | | | | |
| New.ReferenceOTU14 | f_{——}Lachnospiraceae; g_{——}; s_{——} | 0 | 0.0032% | 0.1367% | 0.1275% |
| 128297 | f_{——}Ruminococcaceae; g_{——}; s_{——} | 0.0005% | 0.0016% | 0.2267% | 0.1323% |
| 4403506 | f_{——}Ruminococcaceae; g_{——}; s_{——} | 0.0002% | 0.0073% | 0.0545% | 0.4221% |
| New.CleanUp.ReferenceOTU31483 | f_{——}Ruminococcaceae; | 0.0065% | 0.0538% | 0.1150% | 0.0924% |
| | g_{——}Oscillospira; s_{——} | | | | |
| 350242 | f_{——}Lactobacillaceae; | 0.8172% | 1.3726% | 0.0013% | 0.0020% |
| | g_{——}Lactobacillus; s_{——} | | | | |
| 263744 | f_{——}Ruminococcaceae; | 0.0029% | 0.0252% | 0.1030% | 0.0799% |
| | g_{——}Oscillospira; s_{——} | | | | |
| 831641 | o_{——}RF39; f_{——}; g_{——}; s_{——} | 0.0001% | 0.0013% | 0.1902% | 0.1024% |
| New.ReferenceOTU237 | f_{——}Ruminococcaceae; | 0.0001% | 0.0028% | 0.5274% | 0.2404% |
| | g_{——}Faecalibacterium; s_{——}prausnitzii | | | | |
| 1010876 | f_{——}Ruminococcaceae; | 0 | 0.0028% | 0.0807% | 0.1060% |
| | g_{——}Oscillospira; s_{——} | | | | |
| New.ReferenceOTU225 | f_{——}Peptostreptococcaceae; g_{——}; s_{——} | 0.3284% | 0.0363% | 0.0002% | 0 |
| 4362724 | f_{——}Ruminococcaceae; | 0.0001% | 0.0041% | 0.2051% | 0.2026% |
| | g_{——}Oscillospira; s_{——} | | | | |
| 157224 | f_{——}Ruminococcaceae; | 0.0176% | 0.2092% | 9.9518% | 4.1341% |
| | g_{——}Faecalibacterium; s_{——}prausnitzii | | | | |
| 211795 | f_{——}Lachnospiraceae; g_{——}Blautia; s_{——} | 0.0257% | 0.0230% | 0.1196% | 0.1909% |
| New.ReferenceOTU104 | f_{——}Clostridiaceae; g_{——}; s_{——} | 0.3233% | 0.0279% | 0.0003% | 0 |
| New.ReferenceOTU101 | o_{——}RF39; f_{——}; g_{——}; s_{——} | 0.0005% | 0.0018% | 0.1840% | 0.1525% |
| New.ReferenceOTU322 | o_{——}Clostridiales; f_{——}; g_{——}; s_{——} | 0 | 0.0073% | 0.0774% | 0.1528% |
| 1028036 | f_{——}Bacillaceae; g_{——}; s_{——} | 0.0010% | 0.0021% | 0.2489% | 0.4375% |
| 585880 | o_{——}Clostridiales; f_{——}; g_{——}; s_{——} | 0 | 0.0009% | 0.1819% | 0.0534% |
| New.ReferenceOTU98 | f_{——}Ruminococcaceae; g_{——}; s_{——} | 0 | 0.0010% | 0.1541% | 0.1330% |
| New.ReferenceOTU130 | f_{——}Clostridiaceae; g_{——}; s_{——} | 0.2515% | 0.0239% | 0.0005% | 0 |
| 268002 | o_{——}Clostridiales; f_{——}; g_{——}; s_{——} | 0 | 0.0029% | 0.0882% | 0.2001% |
| 4404461 | f_{——}Ruminococcaceae; g_{——}; s_{——} | 0.0001% | 0.0026% | 0.1141% | 0.0804% |
| New.ReferenceOTU323 | f_{——}Lachnospiraceae; g_{——}; s_{——} | 0.0006% | 0.0027% | 0.0928% | 0.0950% |
| 4407604 | o_{——}Lactobacillales; f_{——}; g_{——}; s_{——} | 0.2661% | 0.1740% | 0.0010% | 0.0003% |
| 132991 | f_{——}Ruminococcaceae; | 0.0033% | 0.0351% | 2.7596% | 2.3301% |
| | g_{——}Faecalibacterium; s_{——}prausnitzii | | | | |
| 4472796 | f_{——}Bacteroidaceae; g_{——}Bacteroides; | 0.0004% | 0.0138% | 0 | 2.6351% |
| | s_{——}fragilis | | | | |
| 183932 | f_{——}Ruminococcaceae; g_{——}; s_{——} | 0.0006% | 0.0034% | 0.3385% | 0.1386% |
| New.ReferenceOTU685 | f_{——}Clostridiaceae; g_{——}; s_{——} | 0.7746% | 0.2196% | 0.0004% | 0.0004% |
| 185391 | f_{——}Ruminococcaceae; g_{——}; s_{——} | 0.0014% | 0.0027% | 0.2944% | 0.0773% |
| New.ReferenceOTU341 | f_{——}Ruminococcaceae; | 0 | 0.0007% | 0.1293% | 0.0897% |
| | g_{——}Faecalibacterium; s_{——}prausnitzii | | | | |
| 235065 | o_{——}RF39; f_{——}; g_{——}; s_{——} | 0.0033% | 0.0063% | 0.7310% | 0.4055% |
| 157888 | f_{——}Ruminococcaceae; g_{——}; s_{——} | 0.0001% | 0.0003% | 0.1322% | 0.0583% |
| New.ReferenceOTU339 | f_{——}Turicibacteraceae; | 0.9726% | 0.2562% | 0.0004% | 0.0006% |
| | g_{——}Turicibacter; s_{——} | | | | |
| 137580 | f_{——}Lactobacillaceae; | 0.3254% | 0.9480% | 0.0014% | 0.0022% |
| | g_{——}Lactobacillus; s_{——} | | | | |
| 4447432 | f_{——}Lactobacillaceae; | 1.5239% | 5.7752% | 0.2604% | 0.5831% |
| | g_{——}Lactobacillus; s_{——} | | | | |
| New.ReferenceOTU561 | o_{——}RF39; f_{——}; g_{——}; s_{——} | 0.0003% | 0.0024% | 0.0596% | 0.2882% |
| 297480 | f_{——}Lachnospiraceae; g_{——}Blautia; s_{——} | 0.0314% | 0.0110% | 0.1030% | 0.1470% |
| 170926 | f_{——}Ruminococcaceae; g_{——}; s_{——} | 0.0009% | 0.0096% | 0.8814% | 1.0219% |
| 182245 | f_{——}Lachnospiraceae; | 0.0097% | 0.0065% | 0.3615% | 0.2414% |
| | g_{——}[Ruminococcus]; s_{——} | | | | |
| 339838 | o_{——}RF39; f_{——}; g_{——}; s_{——} | 0.0001% | 0.0025% | 0.1489% | 0.2532% |
| 173900 | f_{——}Lachnospiraceae; g_{——}; s_{——} | 0.0001% | 0.0028% | 0.1847% | 0.1363% |
| 338438 | f_{——}Ruminococcaceae; g_{——}; s_{——} | 0.0000% | 0.0005% | 0.1105% | 0.0784% |
| 4477479 | f_{——}Lachnospiraceae; | 0.0031% | 0.0027% | 0.1859% | 0.3676% |
| | g_{——}[Ruminococcus]; s_{——} | | | | |
| New.ReferenceOTU491 | o_{——}Clostridiales; f_{——}; g_{——}; s_{——} | 0.0002% | 0.0032% | 0.0907% | 0.1530% |
| 661278 | f_{——}Turicibacteraceae; | 4.3825% | 1.3535% | 0.0559% | 0.0300% |
| | g_{——}Turicibacter; s_{——} | | | | |
| 759349 | f_{——}Enterococcaceae; | 0.2933% | 0.2503% | 0.0007% | 0.0009% |
| | g_{——}Enterococcus; s_{——} | | | | |
| 4370912 | f_{——}Peptostreptococcaceae; g_{——}; s_{——} | 1.2383% | 0.5576% | 0.0028% | 0.0036% |
| 183517 | o_{——}Clostridiales; f_{——}; g_{——}; s_{——} | 0.0002% | 0.0004% | 0.1739% | 0.1090% |
| 191273 | f_{——}Lachnospiraceae; | 0.0011% | 0.0053% | 0.1617% | 0.0961% |
| | g_{——}[Ruminococcus]; s_{——} | | | | |
| New.ReferenceOTU163 | f_{——}Lachnospiraceae; g_{——}; s_{——} | 0 | 0.0008% | 0.1341% | 0.0650% |
| New.ReferenceOTU274 | o_{——}Clostridiales; f_{——}; g_{——}; s_{——} | 0.0001% | 0.0005% | 0.1811% | 0.0962% |
| 166911 | f_{——}Lactobacillaceae; | 4.1970% | 7.6987% | 0.0866% | 0.0897% |
| | g_{——}Lactobacillus; s_{——} | | | | |
| 235424 | f_{——}Peptostreptococcaceae; g_{——}; s_{——} | 0.2311% | 0.0797% | 0.0022% | 0.0028% |
| 911808 | f_{——}Streptococcaceae; | 0.3389% | 0.0979% | 0.0003% | 0.0003% |
| | g_{——}Streptococcus; s_{——} | | | | |
| 130763 | f_{——}Ruminococcaceae; g_{——}; s_{——} | 0.0004% | 0.0058% | 0.3376% | 0.2385% |
| 158211 | f_{——}Lachnospiraceae; g_{——}Blautia; | 0.0055% | 0.0162% | 0.1446% | 0.2238% |
| | s_{——}producta | | | | |
| New.CleanUp.ReferenceOTU36322 | f_{——}Lactobacillaceae; | 0.2319% | 0.0363% | 0 | 0 |
| | g_{——}Lactobacillus; s_{——}salivarius | | | | |
| 988932 | o_{——}Clostridiales; f_{——}; g_{——}; s_{——} | 0.0010% | 0.0013% | 0.2320% | 0.0420% |
| 845900 | f_{——}Ruminococcaceae; | 0.0002% | 0.0026% | 0.2122% | 0.1904% |
| | g_{——}Oscillospira; s_{——} | | | | |
| 270030 | f_{——}Ruminococcaceae; g_{——}; s_{——} | 0 | 0.0011% | 0.1163% | 0.0986% |
| 237063 | o_{——}Clostridiales; f_{——}; g_{——}; s_{——} | 0.0057% | 0.0061% | 0.3558% | 0.6654% |
| New.ReferenceOTU9 | f_{——}Turicibacteraceae; | 0.8023% | 0.3553% | 0.0012% | 0.0016% |
| | g_{——}Turicibacter; s_{——} | | | | |
| New.ReferenceOTU112 | f_{——}Peptostreptococcaceae; g_{——}; s_{——} | 0.9019% | 0.4880% | 0.0015% | 0.0027% |
| 2182669 | o_{——}Clostridiales; f_{——}; g_{——}; s_{——} | 0.0005% | 0.0029% | 0.2065% | 0.1297% |
| 183867 | f_{——}Lachnospiraceae; g_{——}; s_{——} | 0.0623% | 0.0395% | 0.3911% | 0.6872% |
| 1141398 | f_{——}Lactobacillaceae; | 3.1369% | 4.8007% | 0.0449% | 0.0414% |
| | g_{——}Lactobacillus; s_{——}salivarius | | | | |
| New.ReferenceOTU583 | o_{——}Clostridiales; f_{——}; g_{——}; s_{——} | 0.0001% | 0.0006% | 0.1716% | 0.1109% |
| 128227 | f_{——}Lactobacillaceae; | 0.4154% | 0.8363% | 0.0026% | 0.0028% |
| | g_{——}Lactobacillus; s_{——} | | | | |
| 339121 | f_{——}Ruminococcaceae; g_{——}; s_{——} | 0.0000% | 0.0000% | 0.3053% | 0.1393% |
| 288810 | f_{——}Ruminococcaceae; g_{——}; s_{——} | 0.0076% | 0.0153% | 0.2925% | 0.3330% |
| New.ReferenceOTU604 | o_{——}Lactobacillales; f_{——}; g_{——}; s_{——} | 0.8129% | 0.2026% | 0.0004% | 0.0001% |
| New.ReferenceOTU406 | o_{——}RF39; f_{——}; g_{——}; s_{——} | 0.0009% | 0.0022% | 0.2498% | 0.1027% |
| 247639 | f_{——}Clostridiaceae; g_{——}SMB53; s_{——} | 0.7071% | 0.5789% | 0.0012% | 0.0016% |
| 564334 | f_{——}Ruminococcaceae; | 0 | 0.0016% | 0.1758% | 0.2224% |
| | g_{——}Oscillospira; s_{——} | | | | |
| New.ReferenceOTU402 | f_{——}Ruminococcaceae; g_{——}; s_{——} | 0.0001% | 0.0005% | 0.2169% | 0.0826% |
| New.ReferenceOTU648 | f_{——}Ruminococcaceae; | 0.0000% | 0.0008% | 0.3327% | 0.2295% |
| | g_{——}Faecalibacterium; s_{——}prausnitzii | | | | |
| 357765 | c_{——}Clostridia; o_{——}Clostridiales; f_{——}; | 0.0138% | 0.0059% | 0.2819% | 0.0422% |
| | g_{——}; s_{——} | | | | |
| New.ReferenceOTU45 | f_{——}Streptococcaceae; | 0.4441% | 1.0371% | 0.0071% | 0.0136% |
| | g_{——}Streptococcus; s_{——}alactolyticus | | | | |
| 4070490 | f_{——}Ruminococcaceae; g_{——}; s_{——} | 0.0012% | 0.0035% | 0.1068% | 0.1812% |
| 158037 | f_{——}Ruminococcaceae; g_{——}; s_{——} | 0.0009% | 0.0100% | 0.6719% | 0.2344% |
| 4337090 | f_{——}Streptococcaceae; | 0.3260% | 0.6122% | 0.0015% | 0.0012% |
| | g_{——}Streptococcus; s_{——} | | | | |
| 4334055 | o_{——}Lactobacillales; f_{——}; g_{——}; s_{——} | 0.6092% | 0.7234% | 0.0008% | 0.0010% |
| 157704 | o_{——}Clostridiales; f_{——}; g_{——}; s_{——} | 0.0003% | 0.0018% | 0.1042% | 0.0923% |
| New.ReferenceOTU296 | f_{——}Ruminococcaceae; g_{——}; s_{——} | 0.0000% | 0.0005% | 0.1331% | 0.1033% |
| New.ReferenceOTU632 | f_{——}Lactobacillaceae; | 0.2140% | 0.0309% | 0.0001% | 0 |
| | g_{——}Lactobacillus; s_{——} | | | | |
| 3141342 | f_{——}Lachnospiraceae; | 0.0005% | 0.0040% | 0.1823% | 0.1789% |
| | g_{——}Coprococcus; s_{——} | | | | |
| 608244 | f_{——}Ruminococcaceae; | 0.0007% | 0.0057% | 0.2338% | 0.1573% |
| | g_{——}Ruminococcus; s_{——} | | | | |
| 2272797 | f_{——}Turicibacteraceae; | 0.7959% | 0.3138% | 0.0061% | 0.0031% |
| | g_{——}Turicibacter; s_{——} | | | | |
| 687185 | f_{——}Aerococcaceae; g_{——}Aerococcus; | 0.1532% | 0.0578% | 0.0014% | 0.0010% |
| | s_{——} | | | | |
| 4366089 | f_{——}Ruminococcaceae; | 0.0004% | 0.0028% | 0.2669% | 0.1482% |
| | g_{——}Oscillospira; s_{——} | | | | |
| 4405128 | o_{——}YS2; f_{——}; g_{——}; s_{——} | 0.0003% | 0.0005% | 0.2004% | 0.0507% |
| 3421266 | f_{——}Lachnospiraceae; | 0.0007% | 0.0002% | 0.1232% | 0.0945% |
| | g_{——}[Ruminococcus]; s_{——} | | | | |
| New.ReferenceOTU480 | f_{——}Lactobacillaceae; | 0.1003% | 0.5054% | 0.0046% | 0.0047% |
| | g_{——}Lactobacillus; s_{——} | | | | |
| New.ReferenceOTU129 | f_{——}Corynebacteriaceae; | 0.1808% | 0.0584% | 0.0003% | 0 |
| | g_{——}Corynebacterium; s_{——}stationis | | | | |
| New.ReferenceOTU437 | f_{——}Streptococcaceae; | 0.0288% | 0.3835% | 0.0031% | 0.0031% |
| | g_{——}Streptococcus; s_{——} | | | | |
| 234951 | o_{——}RF39; f_{——}; g_{——}; s_{——} | 0.0007% | 0.0031% | 0.1696% | 0.1393% |
| 130773 | f_{——}Lachnospiraceae; | 0.0307% | 0.0214% | 0.6923% | 0.4011% |
| | g_{——}[Ruminococcus]; s_{——} | | | | |
| 157081 | f_{——}Ruminococcaceae; g_{——}; s_{——} | 0.0023% | 0.0087% | 0.6245% | 0.5805% |
| 302823 | f_{——}Ruminococcaceae; | 0.0001% | 0.0014% | 0.1115% | 0.0964% |
| | g_{——}Ruminococcus; s_{——} | | | | |
| New.ReferenceOTU117 | f_{——}Lactobacillaceae; | 0.2139% | 0.2739% | 0.0017% | 0.0028% |
| | g_{——}Lactobacillus; s_{——} | | | | |
| New.ReferenceOTU476 | f_{——}Ruminococcaceae; | 0.0002% | 0.0002% | 0.1869% | 0.0860% |
| | g_{——}Oscillospira; s_{——} | | | | |
| New.ReferenceOTU52 | f_{——}Enterococcaceae; | 0.2565% | 0.0248% | 0.0001% | 0.0003% |
| | g_{——}Enterococcus; s_{——} | | | | |
| New.ReferenceOTU615 | f_{——}Ruminococcaceae; g_{——}; s_{——} | 0 | 0.0005% | 0.1363% | 0.1042% |
| 838685 | f_{——}Ruminococcaceae; | 0.0001% | 0.0026% | 0.1584% | 0.2029% |
| | g_{——}Oscillospira; s_{——} | | | | |
| New.ReferenceOTU592 | o_{——}RF39; f_{——}; g_{——}; s_{——} | 0.0023% | 0.0037% | 0.2516% | 0.1855% |
| New.ReferenceOTU414 | k_{——}Archaea; p_{——}Euryarchaeota; | 2.5436% | 1.7903% | 1.4779% | 0.0086% |
| | c_{——}Methanobacteria; | | | | |
| | o_{——}Methanobacteriales; f_{——}; g_{——}; s_{——} | | | | |
| 157837 | o_{——}Clostridiales; f_{——}; g_{——}; s_{——} | 0.0013% | 0.0013% | 0.2205% | 0.1056% |
| 234421 | f_{——}Lachnospiraceae; | 0.0026% | 0.0114% | 0.1718% | 0.1040% |
| | g_{——}[Ruminococcus]; s_{——} | | | | |
| 4454531 | f_{——}Enterobacteriaceae; g_{——}; s_{——} | 0.0671% | 1.3380% | 0.0789% | 0.1762% |
| New.ReferenceOTU544 | f_staphylococcaceae; | 0.1937% | 0.0453% | 0.0001% | 0.0002% |
| | g_{——}Staphylococcus; s_{——} | | | | |
| 258148 | f_{——}Lachnospiraceae; g_{——}; s_{——} | 0.0005% | 0.0294% | 0.2222% | 0.4041% |
| 131559 | f_{——}Lachnospiraceae; g_{——}Blautia; s_{——} | 0.0136% | 0.0046% | 0.1566% | 0.0380% |
| 4447567 | o_{——}Lactobacillales; f_{——}; g_{——}; s_{——} | 0.4486% | 1.0335% | 0.0012% | 0.0013% |
| 199403 | f_{——}Coriobacteriaceae; | 0.0130% | 0.0047% | 0.2857% | 0.0854% |
| | g_{——}Adlercreutzia; s_{——} | | | | |
| New.ReferenceOTU547 | f_{——}Ruminococcaceae; | 0.0001% | 0.0053% | 0.1280% | 0.1474% |
| | g_{——}Ruminococcus; s_{——} | | | | |
| 237438 | f_{——}Ruminococcaceae; g_{——}; s_{——} | 0.0010% | 0.0124% | 0.2049% | 0.2628% |
| 174651 | f_{——}Ruminococcaceae; g_{——}; s_{——} | 0 | 0.0015% | 0.1488% | 0.1118% |
| New.ReferenceOTU258 | f_{——}Lachnospiraceae; g_{——}; s_{——} | 0.3281% | 0.2566% | 0.0008% | 0.0006% |
| 157470 | f_{——}Lachnospiraceae; g_{——}; s_{——} | 0.0005% | 0.0074% | 0.2682% | 0.2175% |
| 158047 | f_staphylococcaceae; | 0.1472% | 0.0557% | 0.0022% | 0.0009% |
| | g_{——}Staphylococcus; s_{——} | | | | |
| 3323110 | f_{——}Bacteroidaceae; g_{——}Bacteroides; | 0.0006% | 0.4603% | 0.0002% | 13.5961% |
| | s_{——} | | | | |
| 4385535 | f_{——}Bacillaceae; g_{——}Bacillus; s_{——} | 0 | 0.4485% | 0.0000% | 0.0042% |
| New.ReferenceOTU227 | f_{——}Lachnospiraceae; g_{——}Blautia; s_{——} | 0.0196% | 0.0039% | 0.3468% | 0.0852% |
| New.ReferenceOTU493 | f_{——}Ruminococcaceae; g_{——}; s_{——} | 0 | 0.0018% | 0.1321% | 0.0780% |
| 157193 | o_{——}Clostridiales; f_{——}; g_{——}; s_{——} | 0.0011% | 0.0085% | 0.2283% | 0.1686% |
| New.ReferenceOTU281 | f_{——}Streptococcaceae; | 0.1631% | 0.1582% | 0.0002% | 0.0002% |
| | g_{——}Streptococcus; s_{——} | | | | |
| 176615 | f_{——}Lactobacillaceae; | 0.0715% | 0.1466% | 0.0013% | 0.0013% |
| | g_{——}Lactobacillus; s_{——} | | | | |
| New.ReferenceOTU177 | o_{——}RF39; f_{——}; g_{——}; s_{——} | 0.0007% | 0.0017% | 0.2090% | 0.0609% |
| 158360 | f_{——}Ruminococcaceae; g_{——}; s_{——} | 0.0023% | 0.0067% | 0.0397% | 0.1792% |
| 592649 | o_{——}Clostridiales; f_{——}; g_{——}; s_{——} | 0.0005% | 0.0047% | 0.2034% | 0.1190% |
| 2066056 | f_{——}Ruminococcaceae; g_{——}; s_{——} | 0.0008% | 0.0023% | 0.2973% | 0.1295% |
| New.ReferenceOTU619 | f_{——}Clostridiaceae; g_{——}SMB53; s_{——} | 1.1258% | 0.4993% | 0.0014% | 0.0008% |
| New.ReferenceOTU183 | f_{——}Ruminococcaceae; g_{——}; s_{——} | 0.0003% | 0.0018% | 0.1566% | 0.1290% |
| 158309 | f_{——}Ruminococcaceae; g_{——}; s_{——} | 0.0005% | 0.0033% | 0.3712% | 0.2161% |
| 306306 | f_{——}Lactobacillaceae; | 0.5029% | 0.1362% | 0.0050% | 0.0091% |
| | g_{——}Lactobacillus; s_{——} | | | | |
| 592901 | f_{——}Lachnospiraceae; | 0.0087% | 0.0204% | 0.1341% | 0.1649% |
| | g_{——}[Ruminococcus]; s_{——} | | | | |
| New.ReferenceOTU546 | f_{——}Clostridiaceae; g_{——}SMB53; s_{——} | 1.2304% | 0.8142% | 0.0016% | 0.0017% |

### Example 4

### The Probiotic Performance of Bacillus amyloliquefaciens Strain H57 in Pregnant Ewes Fed a Diet Based on Palm Kernel Meal

### Materials and Methods

### Experimental Animals, Treatment and Design

The animals and the experimental procedures were approved by the Animal Ethics Committee of the University of Queensland. The experiment was conducted at the Queensland Animal Science Precinct (QASP) from 24 May 2013 to 14 Nov. 2014 in a large shed with individual animal pens which contained rubber matting on the floor, a bucket for fresh water and a feed container.

Thirty-two first parity white Dorper ewes (day 37 after artificial insemination, mean weight 47.3 kg, mean age 15 months) were relocated into individual pens in animal house at the Queensland Animal Science Precinct (QASP), Gatton. Two weeks before being relocated to QASP, sheep were introduced to the pelleted diet (c. 200 g/d/head) on the stud farm. On the day of arrival, 52 ewes were placed in pens and fed about 800 g/d pellets (no probiotic added) and 400 g/d oaten chaff. The following week, sheep were fed 1-1.2 kg/d pellets and 100 g/d chaff. The period of adjustment in pregnancy (day 37-89) extended beyond expectations due to some evidence of mild ruminal acidosis such as wet faeces and lameness of some ewes. In addition, after some initially high intakes were followed by low intakes. During that time the diet was modified, in an attempt to improve palatability, by the addition of oaten chaff and the removal of an acidifying agent (NH₄Cl) which was added to the pellets to control urinary calculi in sheep. Eight ewes were removed due to poor appetite, leaving 24 ewes to start the trial at day 90 of pregnancy. From day 90 of pregnancy till day 7 post-partum, ewes were fed pellets diet, plus 100 g/ewe/day oaten chaff.

During adjustment period, all ewes were injected subcutaneously with “Cydectin long acting injection for sheep” (Virbac, Australia) for the control of roundworm, nasal bot, itchmite and Haemonchus contortus in sheep and vaccinated with “Vaccin Glanvac 6” (Zoetis, Australia) to protect against Cheesy Gland (CLA) and the five main clostridial diseases; black disease, black leg, malignant oedema, pulpy kidney, and tetanus.

The display of copper toxicity of all ewes after birth led to a decrease in pellets from 90 to 60%, and an increase in hay allowance to 40%. At day 20 of lactation, the loss of one ewe from the Treatment group as a result of copper toxicity led to changing the diet from pellets to a 50:50 mix of lucerne and oaten hay, fed ad libitum, plus 100 g/ewe/day of ground sorghum. The sorghum was used to deliver the H57 dose (4.3×10⁹ cfu/ewe/day) for the treatment ewes.

Sheep were fed to meet 100% of their energy and protein requirements (Freer, 2007) plus 70 g average daily weight gain during pregnancy, and ad libitum during lactation. The amount of feed was calculated for individual ewes depending on their live weight, and number of fetuses. Feed offered was adjusted weekly during the progress of the pregnancy. The sheep were fed twice daily in equal portions at 6.30 am and 4.30 pm.

The ingredients and chemical composition of pelleted diets and oaten chaff are presented in Table 14. The H57 probiotic inoculum was produced in the pilot fermentation plant at the University of Queensland, Gatton Campus. The bacteria (as spores), were mixed in a food grade bentonite carrier and freeze dried. This inoculum was then mixed in a concrete mixer with finely ground sorghum (approximately 1 mm) and 100 kg of mixture was commercially combined with other feed ingredients by Ridley AgriProducts Pty Ltd., to form the pelleted treatment diet. This represented 11% of the final amount of sorghum added to the 2 t batch mix for pelleting. A similar amount of the sorghum grain fines was added to the control pellets. The inoculum supplied sufficient B. amyloliquefaciens H57 spores to give a titre of 2.85×10⁹ cfu/kg of pellet.

TABLE 14
Ingredient and chemical composition of experimental feed

| | Pregnancy diet | Lactation diet |
|---|---|---|
| Ingredients (% DM) | | |
| Palm kernel meal | 37.5 | — |
| Sorghum grain, ground | 39.6 | 4.0 |
| Chickpea hull | 9.5 | — |
| Urea | 0.3 | — |
| Oaten chaff | 8.0 | 48.0 |
| Lucerne chaff | — | 48.0 |
| Molasses | 2.5 | — |
| Limestone | 1.5 | — |
| Salt | 0.5 | — |
| Ammonium sulphate | 0.5 | — |
| Mineral/Vitamin premix^{A} | 0.2 | — |
| H57spores (cfu/kg DM) | +/−2.85 × 10⁹ | +/−4.3 × 10¹⁰ |
| Composition (% DM) | | |
| DM (%) | 91.1 | 83.1 |
| CP | 12.7 | 13.7 |
| OM | 93.5 | 88.6 |
| NDF | 36.8 | 45.3 |
| ADF | 24.7 | 28.5 |
| Lignin | 7.15 | 5.62 |
| Calcium | 10.2 | 9.90 |
| Phosphorus | 3.31 | 3.61 |

DM: dry matter;
CP: crude protein;
NDF: neutral detergent fibre;
ADF: acid detergent fibre

### Feed Intake and Live Weight Change

Animals were weighed weekly to determine live weight change. Feed intake was measured every week by subtracting the feed residues from feed offered. Feed offered for individual ewe was weighed out weekly and fed in daily equal portions, feed residue for each sheep was daily collected and was weighed at the end of the week.

### Digestibility and Nitrogen Retention

Total collection trials were conducted during adjustment period from day 77 to 90 and when sheep were in week 4 of treatment (day 111 to 121 of pregnancy). Sheep were kept in individual metabolism crates for ten days each time, with the first three days for adaptation to the metabolism creates and seven days of total collection.

Diets of both pellets and oaten chaff for individual sheep were prepared at the beginning of the trial, and stored in paper bags. Feed residue, faeces and urine output of individual sheep were measured and sampled daily. About 10% total daily weight of feed residue, faeces and urine of each sheep was taken and stored in a 4° C. room during seven days of collection. For urine samples, approximately 80-100 ml of 5% H₂SO₄ was added into each urine bucket at the start of each daily collection to keep the urine pH just below 3.0 to stabilize the ammonia in the urine. At the end of the collection period daily feed residue, faeces and urine samples were mixed, and two sub-samples of each were taken for each sheep to store at −20° C. for later chemical analysis.

### Rumen Parameters

Rumen fluid was collected during pregnancy at day 90 (pretreated period) and day 126 and day and 63 of lactation. Rumen fluid was collected by a stomach tube at 6 am before morning feeding. Ruminal pH was measured using a portable pH meter immediately on fresh fluid after collection; two sub-samples (4 ml each) of rumen fluid were added to a tube with 1 ml of 20% metaphosphoric acid for volatile fatty acids (VFAs) analysis, and another tube with 2 ml of 20% sulphuric acid for ammonia (NH₃) analysis. These tubes were stored at −20° C.

### Blood Samples

Blood samples of each ewe were collected at fortnightly intervals during pregnancy, and one hour after term for ewes. Approximately 9 mL of blood was taken by standard jugular vein puncture using a 10 mL syringe, 18 gauge needle and was transferred immediately to a 9 mL lithium-heparin tube, mixed gently and store in ice for 30 minutes before centrifugation. Samples were spun at 3500 rpm for 10 minutes at 4° C. to separate plasma and blood cells. Plasma was transferred to a microcentrifuge tube and stored at −20° C. before analysis.

At lambing time, litter size, lamb sex and lamb birth weight were recorded. Lamb birth weight was taken as soon as the dam completed licking the lambs (within 1 hour after birth).

### Chemical Analysis

Samples of all feed, feed residues, faeces were oven dried to a constant weight at 60° C., and ground through a 1 mm screen (Retsch ZM 200; Haan, Germany) for chemical analysis. Dry matter (DM) of the samples was determined by drying at 105° C. for 48 h. Organic matter (OM) content of the samples was determined after incineration at 550° C. for 8 h in a muffle furnace (Modutemp Pty. Ltd.; Perth, WA, Australia) (AOAC, 1990).

Nitrogen content of feed, feed residue, faeces and urine was determined by the Kjeldahl method using a nitrogen analyser (Kjeltec, 8400 FOSS; Hillerod, North Zealand, Denmark).

Neutral detergent fibre (NDF) and acid detergent fibre (ADF) were determined using an Ankom fibre digestion unit using procedures described by the manufacturer (Ankom Technology; Macedon, N.Y., USA). NDF or ADF of the sample was the residue remaining after one hour digestion in neutral or acid detergent solution. The concentration of NDF or ADF was calculated gravimetrically.

The concentration of ruminal VFAs was determined by gas liquid chromatography (GC17, Shimadzu; Kyoto, Honshu, Japan) using a polar capillary column (ZB-FFAP, Phenomenex; Lane Cove, NSW, Australia). The sample was prepared by precipitating the protein, then addition of an internal standard and dilution to minimize loading on the capillary column since the injection was made in splitless mode. A prepared multi-acid standard was mixed with the protein supernatant and this internal standard used to calibrate the gas chromatograph. Samples were then analysed using the internal standardisation method for calibration.

The ruminal ammonia concentration was determined by distillation using a Buchi 321 distillation unit (Flawill, St. Gallen, Switzerland). Sodium tetraborate was added to buffer the sample at around pH 9.5 and decrease hydrolysis of non ammonia compounds. Ammonia was distilled from the mixture using steam. Boric acid captures the ammonia gas, forming an ammonium-borate complex. Ammonia concentration was calculated after titration against a weak HCl solution of known molarity using a TIM 840 Titration Workstation Manager (Radiometer Analysis SAS, Villeubanne, Cedex, France)

The plasma metabolytes aspartate aminotransferase (AST), glutamate dehydrogenase (GLDH), gamma glutamyl transferrase (GGT), total bilirubin (TBIL), cholesterol (CHOL), creatine phosphokinase (CPK), creatinine, urea; electrolytes, and non-esterified fatty acids (NEFA) were determined on an Olympus AU400 auto-analyser (Beckman Coulter Diagnostic Systems Division; Melville, N.Y.C, USA) using the Beckman recommended methods.

### Microbial Profiling of the Rumen

Sheep rumen fluid samples were collected from 24 pregnant dorper ewes (12× control, 12× treatment) using a stomach tube. The rumen fluid contents were aliquoted into 1 ml aliquots, which were then centrifuged at 13,200 rpm for 10 min. The supernatant was removed and the pellet was frozen in liquid Nitrogen. Pelleted samples were then stored at −80° C. until use.

Total genomic DNA was extracted by physical disruption using a bead beating methodology combined with the QIAamp DNA Mini Kit (Qiagen Inc., Valencia, Calif.) as described by Yu and Forster (2005). DNA concentrations and purity were then determined using the Qubit® dsDNA BR Assay Kit with the Qubit® 2.0 Fluorometer (Invitrogen, Carlsbad, Calif.). DNA concentrations were then diluted to 5 ng/μl for sequencing.

16S rRNA Amplicons were then prepared for sequencing as recommended by Illumina (16S Metagenomic Sequencing Library Preparation methodology). In brief, the V6-V8 region of the 16S rRNA gene was amplified using a universal bacterial-specific primer set with added Illumina adapter sequence (iTAG926F=TCGTCGGCAGCGTCAGATGTGTATAAGAGACAGAAACTYAAAKGAATTGR CGG; and iTAG1392wR GTCTCGTGGGCTCGGAGATGTGTATAAGAGACAGACGGGCGGTGWGTRC). The amplified 16S rRNA amplicons were then cleaned in a PCR clean-up step before indices were added by an additional PCR step. The cleaned up product of the index PCR was then denatured then sequenced using the Miseq sequencing platform (Illumina, San Diego, Calif.).

16S rRNA sequencing data generated by Illumina sequencing was processed using quantitative insights into microbial ecology (QIIME) scripted modules. Sequences were filtered according to length, quality, primer and barcode mismatches, homopolymers and chimera removal. The sequencing reads were then clustered together and operational taxonomic units (OTU) were generated using the Open-reference OTU picking script at a similarity threshold of 97%. An OTU table (Table 18) was then generated detailing the relative abundance of each individual OTU per sample.

### Metagenomic Sequencing of the Rumen

DNA Metagenomic libraries were prepared using the Nextera® DNA Sample Preparation Kit (Illumina, San Diego, Calif.) according to manufacturer instructions. Template DNA (50 ng) for each sample was simultaneously fragmented and tagged using 25 μL of Tagment DNA Buffer (Illumina, San Diego, Calif.) and 5 μL of Tagment DNA Enzyme (Illumina, San Diego, Calif.) in a 50 μL reaction. Tagmentation occurred by incubating for 5 min at 55° C. Tagmented DNA was purified with the successive addition of 250 μL of DNA binding buffer (Zymo Research, Irvine, Calif.), 200 μL of Wash Buffer (Zymo Research, Irvine, Calif.), and 25 μL of Resuspension Buffer (Zymo Research, Irvine, Calif.), with centrifugation steps at 10 000 g for 30 s to remove supernatant between buffer additions. Tagmented DNA was indexed and amplified by PCR using dual indexing primers.

The PCR reaction consisted of 5 μL both index primers (IS and 17), 15 μL of Nextera PCR Master Mix (Illumina, San Diego, Calif.), 5 μL of PCR Primer Cocktail (Illumina, San Diego, Calif.) and 20 μL of DNA template. Amplification consisted of and initial denaturation of 98° C. for 30 s, followed by 5 cycles that include a denaturation step of 98° C. for 10 s, annealing at 63° C. for 30 s and elongation at 72° C. for 3 min. The PCR product was then cleaned using AMPure XP beads. The clean up consists of adding 30 μL of AMPure XP beads to 50 μL of PCR product. After an incubation of 5 min at room temperature the samples were placed into a magnetic rack for 2 min. After removing the supernatant the beads were washed with 80% ethanol twice then PCR product was resuspended in 27 μL of Resuspension Buffer. The PCR product was assessed for quality control using a 2100 Bioanalyzer (Agilent Technologies, Santa Clara, Calif.). The DNA libraries were pooled then sequenced on the NextSeq 500 sequencing platform (Illumina, San Diego, Calif.).

### Metagenomic Data Analysis

Sequencing pairs were identified and merged using SeqPrep software (seqprep-2013-08-29). With the use of nesoni clip (nesoni version 0.108), adaptor sequences were removed then sequences were assembled into contiguous sequences using the CLC denovo assembler version 7.5.1. Sequencing reads were then mapped to assembled contigs using BamM version 1.3.8. Generated mapping files were then used to group contigs into genome bins with the use of GroopM version 0.3.4 (Imelfort et al., 2014). The assembled genome bins were then checked for quality control using CheckM version 1.0.0, and those genomes meeting the selected threshold of greater than 60% completeness and less than 10% contamination selected for further analysis. The resulting genomes were then taxonomically classified via a concatenated alignment of 99 marker genes. This alignment was then inferred using FastTree version 2.1.7 and visualised using ARB version 6.0.2. The annotation of selected genome bins were performed using the AnnotateM script (https://github.com/fauziharoon/annotateM) and a glycoside hydrolase profile was established using the CAZy (Carbohydrate Activated Enzyme) database.

To identify which genome bins are most likely representative of the dominant OTUs observed from the 16S rRNA amplicon sequencing, the sequencing reads of each animal were mapped against each genome bin using BamM version 1.3.8. The percentage of reads that mapped to each genome bin was determined and compared to the percentage abundance of each dominant OTU and matches were identified.

### Statistical Analysis

Analyses of feed intake, ewe live weight change, plasma parameters and body condition score (BCS), lamb live weight change were conducted using a repeated measure ANOVA in STATISTICA 8. Sum of squares were partitioned into effects for treatment, time, and age of animals along with all possible interactions. Sheep within treatment were included as a random effect and time was considered as a repeated factor. Rumen characteristic, digestibility of pregnant ewes and lamb birth weight were analyzed using one-way ANOVA in STATISTICA 8. The model includes the fixed effect of treatment, the random effect of sheep within treatment and random residual error.

### Results

Twenty three out of 24 ewes in this trial had a single lamb, and one had twins, for consistency, the ewe with twins was not used in any of the statistical analyses.

### Dry Matter Intake

The B. amyloliquefaciens H57 supplement significantly affected feed intake. During the pre-treatment period, from day 43 to day 90 of pregnancy, DMI of ewes in the two groups was similar, but diverged after B. amyloliquefaciens H57 feeding started Probiotic supplemented ewes had a higher DMI (1041 versus 889 g/d, P=0.02) from day 90 of pregnancy to parturition. DMI in the Treatment group remained constant and closed to the amount of feed offered (1180 g/d) from day 98 to 121, then increased by 100 g a week from day 126 to day 133 before a slight decrease for the last two weeks of gestation. The DMI of control ewes marginally decreased from day 98 to 121, then slightly increased before decreasing again during the last four weeks of gestation. Feed intake in both groups increased after lambing. For the first 20 days post partum, ewes were fed 60% pellets with 40% mixed chaff (50 lucerne: 50 oaten) or 100% mixed chaff for the rest of the trial, feed intake was similar between two groups.

### Digestibility and Nitrogen Retention

Probiotic treatment had no effect on the digestibility of DM, OM, NDF and crude protein of the diet (Table 15).

The nitrogen retention between groups was comparable during the pre-treatment, about 2.94 g/d. Nitrogen balance in probiotic-fed ewes was double that in the Control group (6.47 g/d vs 3.03 g/d, P<0.001 Table 15).

### Ewe and Lamb Liveweight Change

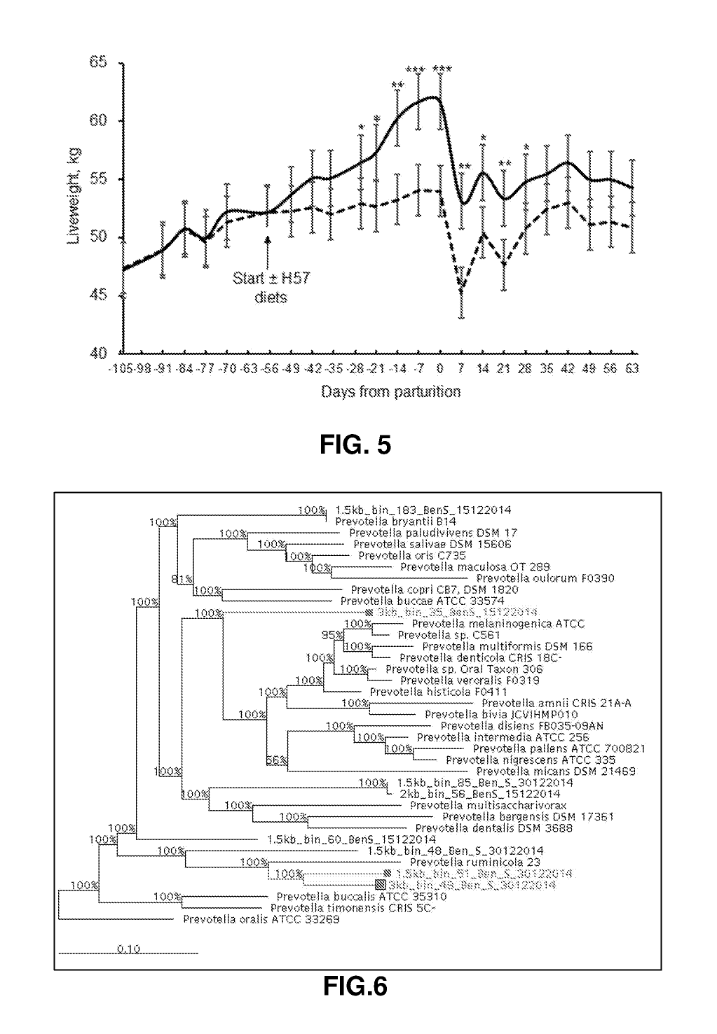

Live weight of ewes in both groups increased over the pregnancy, but at a faster rate with probiotic addition (Table 16, FIG. 5). The average body weight of ewes at beginning of the trial (day 43 after conception) was 47.1±1.9 kg for the Treatment group and 47.3±2.0 kg for the Control. During the 46 days of pre-treatment, ewes in both groups gained an equivalent amount of about 5 kg body weight. At 90 of pregnancy when ewes were started feeding the probiotic B. amyloliquefaciens H57, live weight of the two groups was comparable, 52.2±1.8 kg for the Control and 52.1±1.9 kg for the Treatment. Supplementing with B. amyloliquefaciens H57 had a positive effect on body weight change of pregnant ewes over the next 56 days, with an average 11.3 kg gain compared with 2.1 kg in the Control group. The live weight of treated ewes at parturition was 17% higher than in the Control, (63.6±2.3 kg versus 54.0±2.2 kg, P<0.05).

The lambs of the H57 ewes grew faster than those of the control ewes, but only for the first 21 days of lactation (g/day: 265 vs 356, sed=16.5, P=0.006), but not thereafter. In the current study we particularly highlighted the capacity of H57 to stimulate immature ewes to continue to grow maternal tissue through pregnancy which appeared then to stimulate a greater capacity to partition nutrients to their lambs through milk, at least for the first few weeks of lactation, a critical time for optimising lamb survival. As such, H57 can survive the steam pelleting process to improve the palatability of a diet based on PKM and increase maternal tissue gain in pregnancy to improve ewe performance in early lactation.

### Rumen Fermentation

Supplementing with B. amyloliquefaciens H57 affected some rumen fermentation parameters (Table 17). Rumen pH increased, but total VFAs and ruminal ammonia decreased in ewes receiving probiotic B. amyloliquefaciens H57. Rumen pH in the Treatment was 0.33 units higher than in the Control, (7.11 versus 6.78, P=0.04). Both ruminal total VFAs and ammonia concentration in the Treatment were significantly lower than in the Control group. For molar VFAs, there was no difference in acetate or butyrate, but lower propionate and higher valerate proportion were recorded in treated ewes (P<0.05). Therefore, the ratio of acetate and propionate in ewes receiving B. amyloliquefaciens H57 was higher than in Control ewes, 3.4 versus 2.7, P=0.046.

### Rumen Microbial Changes

The B. amyloliquefaciens H57 supplement produced a number of changes in the microbial flora of the treated ewes. The average relative abundance of each OTU per treatment group is presented in Table 18. Of the control animals the rumen population is dominated by two OTUs of the Prevotella genus, whilst in the +H57 animals the two most dominant OTUs belong to a different OTU of the Prevotella genus as well as a member of the Coprococcus genus (Table 18)

TABLE 15
Effect of B. amyloliquefaciens H57 on digestibility
and nitrogen retention of late pregnant ewes

| | Adjusment | Late pregnancy (day 111-day 121) | | | |
|---|---|---|---|---|---|
| Items | period | H57 | Control | SEM | P value |
| Number of animals | 23 | 10 | 12 | | |
| DMI (g/d) | 904 | 1029 | 837 | | 0.21 |
| Nitrogen intake (g/d) | 18.3 | 20.4 | 17.5 | | 0.10 |
| Urinary nitrogen | 9.39 | 7.89 | 9.45 | | 0.15 |
| excretion (g/d) | | | | | |
| Fecal nitrogen | 5.87 | 6.03 | 4.91 | | 0.17 |
| excretion (g/d) | | | | | |
| Nitrogen retention | | | | | |
| g/d | 2.94 | 6.47 | 3.03 | | 0.001 |
| % nitrogen intake | 15.2 | 29.5 | 18.1 | | |
| Digestibility (%) | | | | | |
| DMD | 64.0 | 68.3 | 66.5 | | 0.20 |
| OMD | 68.4 | 70.8 | 69.6 | | 0.27 |
| NDFD | 45.1 | 47.0 | 45.7 | | 0.36 |
| CPD | 68.2 | 71.1 | 72.7 | 1.83 | 0.40 |

DMI: dry matter intake;
DMD: dry matter digestibility;
OMD: organic matter digestibility;
NDFD: neutral detergent digestibility;
CPD: crude protein digestibility
P77-P87: day 77-87 after conception;
P111-P121: day 111-121 after conception

TABLE 16
Effect of B. amiloliquefaciens H57 supplement
on performance of pregnant and lactating ewes

| Items | | Control | Treatment | sed | P value |
|---|---|---|---|---|---|
| DMI, g/d | | | | | |
| Pregnancy | Day 43-day 89 | 1048 | 1076 | 22.3 | 0.52 |
| | Day 90-day 147 | 889 | 1041 | 42.4 | 0.04 |
| Lactation | Day 0-day 20 | 1480 | 1515 | 28.2 | 0.69 |
| | Day 21-day 63 | 2095 | 2050 | 33.5 | 0.37 |
| Live weight, kg | | | | | |
| Pregnancy | Day 90-147 | 24.0 | 193 | 25.4 | 0.0002 |
| Lactation | Day 0-63 | 97.0 | 1.51 | 21.7 | 0.012 |
| Body condition score (BCS) | | | | | |
| Mid pregnancy | | 3.16 | 3.27 | 0.09 | 0.46 |
| Late pregnancy | | 3.20 | 3.59 | 0.12 | 0.04 |
| Lactation | | 2.40 | 2.75 | 0.09 | 0.015 |
| Gestation length, day | | 146.4 | 147.5 | 0.55 | 0.18 |
| Number of lambs | | 10 | 9 | | |
| Lambs birth weight, kg | | 3.99 | 4.18 | 0.19 | 0.54 |
| Lambs final weight, kg | | 23.2 | 24.5 | 0.70 | 0.24 |
| Lambs | 0-21 day old | 269 | 341 | 15.7 | 0.007 |
| ADG, g/d | 22-63 day old | 296 | 320 | 11.3 | 0.19 |

DMI: dry matter intake;
sed: standard error of the difference;
ADG: average daily gain

TABLE 17
Effect of B. amyloliqiuefaciens H57
on rumen fermentation of pregnant ewes

| Items | Pretreated | H57 | Control | SEM | P value |
|---|---|---|---|---|---|
| Rumen pH | 6.93 | 7.11 | 6.79 | 0.10 | 0.047 |
| Rumen ammonia (mg/l) | 111.1 | 69.1 | 147.6 | 18.2 | 0.006 |
| Total VFAs (mmol/l) | 65.1 | 39.2 | 61.4 | 5.61 | 0.01 |
| Molar VFAs (% total) | | | | | |
| Acetate | 55.0 | 60.9 | 59.2 | 1.7 | 0.55 |
| Propionate | 29.9 | 18.2 | 23.8 | 1.5 | 0.016 |
| n-Butyrate | 11.9 | 17.2 | 14.2 | 1.2 | 0.09 |
| Iso-Butyrate | 1.1 | 0.87 | 0.98 | 0.17 | 0.6 |
| Iso-Valerate | 1.1 | 1.12 | 1.06 | 0.14 | 0.77 |
| n-Valerate | 0.91 | 1.54 | 0.81 | 0.16 | 0.005 |
| A/P ratio | 1.9 | 3.4 | 2.7 | 0.23 | 0.046 |

VFAs: volatile fatty acids;
A/P ratio: acetate/propionate ratio

TABLE 18
The average relative abundance of OTU's generated from sheep rumen fluid.

| | Week 8 | | | Week 13 | | |
|---|---|---|---|---|---|---|
| Taxonomy | Control | +H57 | P-value | Control | +H57 | P-value |
| o_{——}Methanobacteriales | | | | | | |
| f_{——}Methanobacteriaceae; | 1.583% | 1.271% | 0.685 | 6.777% | 2.425% | 0.049* |
| g_{——}Methanobrevibacter; s_{——} | | | | | | |
| o_{——}Bacteroidales | | | | | | |
| f_{——}Paraprevotellaceae; g_{——}; s_{——} | 0.211% | 0.135% | 0.405 | 0.170% | 2.317% | 0.010** |
| f_{——}Prevotellaceae; g_{——}Prevotella; | 0.079% | 0.026% | 0.456 | 1.655% | 0.083% | 0.071 |
| s_{——} | | | | | | |
| f_{——}Prevotellaceae; g_{——}Prevotella; | 0.062% | 0.045% | 0.726 | 0.034% | 1.306% | 0.000** |
| s_{——} | | | | | | |
| f_{——}Prevotellaceae; g_{——}Prevotella; | 9.187% | 2.496% | 0.052 | 0.391% | 19.473% | 0.000** |
| s_{——} | | | | | | |
| f_{——}Prevotellaceae; g_{——}Prevotella; | 0.057% | 0.241% | 0.213 | 1.068% | 0.002% | 0.203 |
| s_{——} | | | | | | |
| f_{——}Prevotellaceae; g_{——}Prevotella; | 1.842% | 1.120% | 0.337 | 0.820% | 1.580% | 0.281 |
| s_{——} | | | | | | |
| f_{——}Prevotellaceae; g_{——}Prevotella; | 0.037% | 0.379% | 0.225 | 2.397% | 0.001% | 0.338 |
| s_{——} | | | | | | |
| f_{——}Prevotellaceae; g_{——}Prevotella; | 1.517% | 1.952% | 0.829 | 1.797% | 0.004% | 0.117 |
| s_{——} | | | | | | |
| f_{——}Prevotellaceae; g_{——}Prevotella; | 7.906% | 8.396% | 0.894 | 8.888% | 0.152% | 0.006** |
| s_{——} | | | | | | |
| f_{——}Prevotellaceae; g_{——}Prevotella; | 1.270% | 0.236% | 0.061 | 0.571% | 0.006% | 0.100 |
| s_{——} | | | | | | |
| f_{——}Prevotellaceae; g_{——}Prevotella; | 19.740% | 12.119% | 0.115 | 12.724% | 1.989% | 0.005** |
| s_{——}ruminicola | | | | | | |
| f_{——}S24-7; g_{——}; s_{——} | 0.252% | 0.200% | 0.538 | 0.504% | 1.640% | 0.026* |
| f_{——}unknown | 0.489% | 0.026% | 0.340 | 1.154% | 0.016% | 0.034* |
| o_{——}Clostridiales | | | | | | |
| f_{——}Eubacteriaceae; | 0.134% | 0.099% | 0.562 | 0.037% | 1.109% | 0.001** |
| g_{——}Pseudoramibacter_Eubacterium; | | | | | | |
| s_{——} | | | | | | |
| f_{——}Lachnospiraceae; g_{——}; s_{——} | 0.142% | 0.109% | 0.784 | 0.223% | 1.696% | 0.130 |
| f_{——}Lachnospiraceae; g_{——}; s_{——} | 0.030% | 0.029% | 0.959 | 0.121% | 1.039% | 0.009** |
| f_{——}Lachnospiraceae; g_{——}Blautia; s_{——} | 1.762% | 1.411% | 0.465 | 0.492% | 4.153% | 0.000** |
| f_{——}Lachnospiraceae; | 0.014% | 0.000% | 0.192 | 0.723% | 4.832% | 0.039* |
| g_{——}Butyrivibrio; s_{——} | | | | | | |
| f_{——}Lachnospiraceae; | 4.604% | 3.700% | 0.537 | 1.563% | 12.172% | 0.000** |
| g_{——}Coprococcus; s_{——} | | | | | | |
| f_{——}Lachnospiraceae; | 0.546% | 0.457% | 0.852 | 0.392% | 1.200% | 0.278 |
| g_{——}Lachnobacterium; s_{——} | | | | | | |
| f_{——}Lachnospiraceae; g_{——}Roseburia; | 0.513% | 0.100% | 0.068 | 0.439% | 3.401% | 0.080 |
| s_{——}faecis | | | | | | |
| f_{——}Lachnospiraceae; | 0.490% | 0.227% | 0.196 | 0.463% | 1.155% | 0.045* |
| g_{——}Shuttleworthia; s_{——} | | | | | | |
| f_{——}unknown | 0.202% | 0.038% | 0.128 | 0.111% | 1.347% | 0.045* |
| f_{——}Veillonellaceae; g_{——}; s_{——} | 0.383% | 0.000% | 0.339 | 2.657% | 0.000% | 0.107 |
| f_{——}Veillonellaceae; | 0.671% | 0.284% | 0.053 | 0.333% | 1.179% | 0.000** |
| g_{——}Acidaminococcus; s_{——} | | | | | | |
| f_{——}Veillonellaceae; | 0.084% | 0.116% | 0.683 | 2.857% | 0.009% | 0.153 |
| g_{——}Selenomonas; s_{——} | | | | | | |
| o_{——}Aeromonadales | | | | | | |
| f_{——}Succinivibrionaceae; g_{——}; s_{——} | 4.622% | 6.485% | 0.428 | 1.878% | 0.496% | 0.267 |
| f_{——}Succinivibrionaceae; g_{——}; s_{——} | 1.412% | 5.870% | 0.346 | 7.243% | 0.202% | 0.122 |
| f_{——}Succinivibrionaceae; g_{——}; s_{——} | 0.310% | 0.000% | 0.339 | 1.010% | 0.000% | 0.302 |
| f_{——}Succinivibrionaceae; | 3.650% | 8.185% | 0.214 | 3.347% | 1.749% | 0.657 |
| g_{——}Ruminobacter; s_{——} | | | | | | |
| f_{——}Succinivibrionaceae; | 7.541% | 10.337% | 0.638 | 1.232% | 0.258% | 0.208 |
| g_{——}Ruminobacter; s_{——} | | | | | | |
| f_{——}Succinivibrionaceae; | 6.804% | 12.478% | 0.197 | 6.217% | 1.724% | 0.064 |
| g_{——}Succinivibrio; s_{——} | | | | | | |

Control: rumen fluid samples collected from ewes that were not fed the probiotic B. amyloliquefaciens H57; +H57: rumen fluid samples collected from ewes fed the probiotic B. amyloliquefaciens H57.

### Rumen Metagenomic Sequencing

High throughput metagenomic sequencing was performed to extract and assemble genomes of dominant organisms within a population. In this instance it was hoped to extract the genomes of the different Prevotella species, which dominated the rumen fluid of each treatment group. Genomes assembled from the control animals are presented in Table 19, while Table 20 shows the genomes assembled from the +H57 animals.

By comparing the percentage of reads that mapped to each genome bin, to the relative abundance of the 16S rRNA amplicon results, it was determined that the closest match for the dominant Prevotella OTU in the control animals is the genome 1.5kb_bin_51 (Table 21). In the +H57 animals the genome most likely representing the dominant Prevotella OTU was 3kb_bin_35 (Table 22). The classification of these genomes has indeed classified them as a part of the Prevotella genus, although where as the control Prevotella classified as the Prevotella ruminicola species, the +H57 Prevotella did not classify with any represented species within the Prevotella genus (FIG. 6).

For a comparison between the Prevotella genome that was shown to be dominant within the control animals and the Prevotella that was dominant in the +H57 animals, the genomes of these organisms were annotated then searched against the CAZy database to develop a profile of glycoside hydrolases (GH) for each genome. The percentage of glycoside hydrolases that were assigned to each GH family is presented in Table 23. The table reveals that the two Prevotella genomes extracted from the control animals (1.5kb_bin_51 and 3kb_bin_49) have a CAZy profile that is predominately composed of GH2 and GH43 families as opposed to the Prevotella genome isolated from the +H57 animals which is dominated by GH5 and GH13 glycoside hydrolases. The GH2 and GH43 families include enzymes that are responsible for the degradation of carbohydrates that constitute hemi-cellulose, the less fibrous portion of the plant cell wall. The GH5 and GH13 families found in the +H57 Prevotella are classified for their role in the degradation of cellulose and starch respectively. The differences in GH profile suggest a more fibrolytic role of the Prevotella that was found to dominate the +H57 animals, as opposed to the hemi-cellulolytic role of the control dominated Prevotella.

TABLE 19
Genome bins assembled from control samples

| | Complete- | | | # |
|---|---|---|---|---|
| Bin Id | ness | Contamination | Length (bp) | Contigs |
| 1.5 kb_bin_69 | 99.21 | 0 | 1986733 | 50 |
| 2 kb_bin_52 | 98.54 | 0.88 | 2366331 | 159 |
| 1.5 kb_bin_85 | 98.35 | 1.07 | 3413580 | 166 |
| 1.5 kb_bin_51 | 97.36 | 8.24 | 3930332 | 595 |
| 1.5 kb_bin_59 | 97.2 | 0.82 | 3816218 | 229 |
| 2 kb_bin_42 | 96.9 | 5.53 | 2755399 | 251 |
| 2 kb_bin_49 | 96.57 | 5.86 | 3799703 | 235 |
| 1.5 kb_bin_46 | 96.29 | 0.57 | 2905293 | 196 |
| 3 kb_bin_49 | 95.91 | 2.71 | 3637604 | 109 |
| 2 kb_bin_9 | 93.97 | 5.24 | 3718458 | 307 |
| 2 kb_bin_76 | 91.25 | 3.62 | 2489772 | 163 |
| 2 kb_bin_21 | 90.54 | 4.76 | 3658785 | 120 |
| 1.5 kb_bin_48 | 89.04 | 6.66 | 2799163 | 445 |
| 3 kb_bin_53 | 81.99 | 6.49 | 2062149 | 226 |

TABLE 20
Genome bins assembled from +H57 samples

| | Complete- | | | # |
|---|---|---|---|---|
| Bin Id | ness | Contamination | Length (bp) | Contigs |
| 1.5 kb_bin_132 | 100 | 1.81 | 2779499 | 58 |
| 2 kb_bin_134 | 100 | 6.51 | 2882788 | 262 |
| 1.5 kb_bin_65 | 100 | 0 | 2087249 | 33 |
| 2 kb_bin_56 | 99.64 | 1.45 | 3742818 | 88 |
| 1.5 kb_bin_60 | 99.64 | 1.45 | 3742818 | 88 |
| 1.5 kb_bin_87 | 97.74 | 8.62 | 3405976 | 225 |
| 2 kb_bin_48 | 96.99 | 0.23 | 2902797 | 172 |
| 1.5 kb_bin_110 | 96.74 | 5.9 | 3328639 | 227 |
| 3 kb_bin_35 | 96.63 | 0.23 | 2967989 | 195 |
| 2 kb_bin_38 | 93.74 | 0.73 | 2949274 | 374 |
| 3 kb_bin_81 | 91.3 | 2.82 | 3010112 | 161 |
| 1.5 kb_bin_61 | 88.35 | 1.67 | 2970249 | 131 |
| 2 kb_bin_87 | 86.94 | 6.92 | 2290262 | 404 |
| 3 kb_bin_77 | 84.16 | 7.41 | 1688815 | 226 |
| 3 kb_bin_49 | 83.13 | 6.1 | 2457945 | 548 |
| 1.5 kb_bin_76 | 83.1 | 2.51 | 2008635 | 177 |
| 2 kb_bin_117 | 70.24 | 4.67 | 1389620 | 276 |
| 3 kb_bin_113 | 69.05 | 0 | 1247520 | 23 |
| 1.5 kb_bin_52 | 68.39 | 7.94 | 1866974 | 252 |
| 1.5 kb_bin_183 | 65.33 | 3.67 | 2099223 | 301 |
| 2 kb_bin_32 | 64.04 | 0 | 1875281 | 100 |
| 3 kb_bin_45 | 62.19 | 2.84 | 1270311 | 108 |
| 1.5 kb_bin_72 | 60.46 | 1.65 | 1258448 | 84 |

TABLE 21
Relative abundance of dominant OTUs compared to percentage of reads mapped
to assembled genome bins from animals within the control group.

| | | | ewe | ewe | ewe | ewe | ewe | ewe |
|---|---|---|---|---|---|---|---|---|
| | OTU | Top Blast Hit | 397 | 470 | 613 | 641 | 652 | 681 |
| ♦ | 807321 | g_{——}Prevotella; | 16.89% | 21.90% | 14.26% | 0.02% | 15.27% | 6.82% |
| | | s_{——} | | | | | | |
| | 542536 | g_{——}Prevotella; | 6.40% | 1.12% | 15.02% | 27.98% | 6.43% | 19.80% |
| | | s_{——}ruminicola | | | | | | |
| | 533298 | g_{——}Succinivibrio; | 8.65% | 2.93% | 10.01% | 14.91% | 4.75% | 14.83% |
| | | s_{——} | | | | | | |

| | | ewe | ewe | ewe | ewe | ewe | ewe |
|---|---|---|---|---|---|---|---|
| | Bin Id | 397 | 470 | 613 | 641 | 652 | 681 |
| | 1.5 kb_bin_46 | 0.01% | 20.44% | 0.02% | 0% | 18.66% | 6.32% |
| | 1.5 kb_bin_48 | 0.57% | 1.57% | 0.34% | 0.08% | 1.84% | 1.11% |
| ♦ | 1.5 kb_bin_51 | 18.23% | 26.93% | 23.44% | 3.13% | 32.77% | 12.31% |
| | 1.5 kb_bin_59 | 0.06% | 6.87% | 11.11% | 0.04% | 8.49% | 2.09% |
| | 1.5 kb_bin_69 | 0.35% | 0.34% | 1.5% | 1.66% | 1.72% | 3.42% |
| | 1.5 kb_bin_85 | 7.97% | 2.17% | 0.06% | 0.14% | 0.01% | 0.01% |
| | 2 kb2_bin_21 | 0.06% | 0.01% | 0.02% | 0.03% | 0.01% | 6.83% |
| | 2 kb2_bin_42 | 0.03% | 6.26% | 0.02% | 0.02% | 0.03% | 0.25% |
| | 2 kb2_bin_49 | 12.66% | 0.04% | 1.61% | 0.07% | 0.06% | 0.04% |
| | 2 kb2_bin_52 | 5.14% | 0.38% | 0.06% | 0.11% | 0.16% | 0.07% |
| | 2 kb2_bin_76 | 3.85% | 2.27% | 0.33% | 1.4% | 0.82% | 0.64% |
| | 2 kb2_bin_9 | 4.27% | 0.01% | 0.05% | 0.03% | 0.01% | 0.03% |
| | 3 kb_bin_49 | 1.72% | 4.16% | 1.05% | 0.85% | 8.38% | 6.09% |
| | 3 kb_bin_53 | 1.79% | 0.1% | 0.97% | 0.88% | 0.18% | 0.04% |
| | unbinned | 43.28% | 28.44% | 59.42% | 91.56% | 26.86% | 60.76% |

Symbol identifies closest matching genome to respective OTU identified in 16S amplicon sequencing.

TABLE 22
Relative abundance of dominant OTUs compared to percentage of reads
mapped to assembled genome bins from animals within the +H57 group.

| | | | ewe | ewe | ewe | ewe | ewe | ewe |
|---|---|---|---|---|---|---|---|---|
| | OTU ID | Taxonomy | 391 | 457 | 611 | 633 | 748 | 771 |
| ♦ | 216587 | g_{——}Prevotella | 12.81% | 30.18% | 15.36% | 28.50% | 16.26% | 9.32% |
| | 4463709 | g_{——}Coprococcus | 16.81% | 12.23% | 12.87% | 11.51% | 2.42% | 12.00% |
| ▪ | 818289 | g_{——}Butyrivibrio | 1.28% | 5.49% | 1.50% | 6.92% | 2.33% | 21.56% |
| | 195186 | g_{——}Blautia | 5.47% | 3.96% | 4.20% | 5.08% | 0.71% | 4.02% |

| | | ewe | ewe | ewe | ewe | ewe | ewe |
|---|---|---|---|---|---|---|---|
| | Bin Id | 391 | 457 | 611 | 633 | 748 | 771 |
| | 1.5 kb_bin_110 | 0.38% | 0.48% | 1.89% | 0.29% | 2.17% | 1.11% |
| | 1.5 kb_bin_132 | 6.08% | 3.6% | 9.61% | 3.36% | 0.15% | 5.82% |
| | 1.5 kb_bin_183 | 0.05% | 0.3% | 0.27% | 0.67% | 0.25% | 0.13% |
| | 1.5 kb_bin_52 | 1.03% | 0.02% | 0.04% | 0.02% | 0.18% | 0.03% |
| | 1.5 kb_bin_61 | 2.57% | 1.71% | 3.51% | 2.96% | 0.97% | 9.51% |
| | 1.5 kb_bin_65 | 5.15% | 0.38% | 2.67% | 2.47% | 0.02% | 17.35% |
| | 1.5 kb_bin_87 | 3.99% | 1.16% | 8.39% | 1.86% | 16.35% | 2.94% |
| ▪ | 2 kb_bin_38 | 0.8% | 4.54% | 1.5% | 5.84% | 0.96% | 14.52% |
| | 2 kb_bin_48 | 0.1% | 0.18% | 1.02% | 0% | 0.03% | 0.33% |
| | 2 kb_bin_53 | 7.35% | 11.29% | 8.83% | 7.39% | 23.6% | 2.52% |
| | 2 kb_bin_56 | 5.37% | 6.4% | 1.7% | 1.15% | 0.06% | 1.4% |
| | 2 kb_bin_87 | 0.75% | 0.16% | 0.78% | 0.27% | 3.55% | 0.25% |
| | 3 kb_bin_113 | 0.41% | 0.33% | 0.84% | 0.17% | 0.04% | 0.52% |
| ♦ | 3 kb_bin_35 | 12.14% | 23.24% | 16.06% | 30.87% | 16.52% | 10.56% |
| | 3 kb_bin_45 | 0.36% | 0.45% | 0.59% | 0.44% | 0.12% | 0.49% |
| | 3 kb_bin_49 | 5.71% | 5.04% | 6.94% | 5.64% | 0.97% | 5.8% |
| | 3 kb_bin_77 | 0.85% | 2.29% | 0.64% | 0.86% | 0.03% | 0.1% |
| | 3 kb_bin_81 | 0.45% | 0.38% | 2.6% | 3.66% | 0.04% | 0.1% |
| | unbinned | 44.79% | 34.15% | 28.84% | 30.61% | 30.31% | 24.52% |

Symbols identify closest matching genome to respective OTU from 16S amplicon sequencing

TABLE 23
Glycoside hydrolase profile of Prevotella
genomes extracted from metagenomic sequencing

| | CAZy | | Control | | +H57 |
|---|---|---|---|---|---|
| | Family | Known Activity | 1.5 kb_bin_51 | 3 kb_bin_49 | 3 kb_bin_35 |
| | Cellulases | | | | |
| ♦ | GH5 | cellulase | 7.92% | 6.67% | 11.59% |
| | GH9 | endoglucanase | 1.98% | 0.95% | 2.9% |
| | Total | | 9.9% | 7.62% | 14.49% |
| | Oligosaccharide-degrading enzymes | | | | |
| ▪ | GH2 | β-galactosidases and other | 8.91% | 11.43% | 5.8% |
| | | β-linked dimers | | | |
| | GH3 | mainly β-glucosidases | 5.94% | 4.76% | 2.9% |
| | GH31 | α-glucosidases | 4.95% | 3.81% | 2.9% |
| | GH35 | β-galactosidase and exo-β- | 0.99% | 1.9% | 1.45% |
| | | glucosaminidase | | | |
| | GH37 | α-trehalase | 0.99% | 0% | 0% |
| ▪ | GH43 | arabinases and xylosidases | 10.89% | 16.19% | 2.9% |
| | GH97 | α-glucosidase and α- | 3.96% | 3.81% | 2.9% |
| | | galactosidase | | | |
| | Total | | 40.59% | 41.9% | 20.3% |
| | Amylases | | | | |
| ♦ | GH13 | α-amylases | 4.95% | 4.76% | 10.14% |
| | GH57 | α-amylases | 0.99% | 0.95% | 1.45% |
| | GH77 | amylomaltase | 1.98% | 1.9% | 2.9% |
| | Total | | 7.92% | 7.61% | 14.49% |

| | | | |
|---|---|---|---|
| Total GH hits: | 101 | 105 | 69 |
| Total ORFs: | 3373 | 2983 | 2330 |
| % GH ORFs: | 2.99% | 3.52% | 2.96% |

♦ = Dominant glycoside hydrolases in +H57 Prevotella;
▪ = Dominant glycoside hydrolases in control Prevotella

### Discussion

An increase in feed intake is considered an important strategy to improve ruminant production. Mean feed intake over the 56 days feeding with B. amyloliquefaciens H57 during late pregnancy was 1041 g/d, close to the amount of feed offered (1180 g/d DM), but was much lower in the control group with only 889 g/d average intake. This result was similar to that reported by Kowalski et al. (2009), with a 12.6% increase in the intake of a starter diet by calves fed a probiotic containing spores of Bacillus licheniformis and Bacillus subtilis. Similarly, calves supplemented with a probiotic mixture containing several Lactobacillus spp., had a significantly increased feed intake, 7.0 kg/week for treated calves compared to 3.7 kg/week for the control group (Frizzo et al., 2012).

As a consequence of increased feed intake which increased exogenous nutrients for the growth of the dam, live weight in the H57 treated group increased by 11.3 kg by parturition compared to 2.1 kg in the control group. Improvement in live weight and daily weight gain by addition of a probiotic were also recorded in young calves (Adams et al., 2008; Sun et al., 2010; Timmerman et al., 2005) and in finishing lambs (Khalid et al., 2011). Diverging from this trend, Kritas et al. (2006) supplemented the diet of pregnant ewes, given 4-6 hours access to pasture, with pellets containing Bacillus licheniformis and Bacillus subtilis and found no response in feed intake or live weight change, but found a positive effect on milk yield, milk fat and milk protein in the probiotic supplemented ewes.

The addition of B. amyloliquefaciens H57 to the pregnancy diet had no effect on the digestibility of nutrients. The digestibility of the dietary nutrients was comparable between the two groups and between the two measurement times before and after supplementation of probiotic. The digestibility of DM, OM and CP of the experimental diet was similar to those found by O'Mara et al. (1999) and Carvalho et al. (2005), for diets that contained similar levels of PKM fed to sheep at a maintenance level. However, the digestibility of NDF in this current experiment was lower than in other reports. NDFD of dietary in this trial was less than 50% in both Treatment and Control groups, whereas, this was 65.6% for diet containing 45%-70% PKM with 20% molasses and 20% grass hay (O'Mara et al. (1999) and 59.1% for diet containing 45% PKM with dehydrated alfalfa (Carvalho et al. (2005). The combination of PKM and a high level of sorghum grain, may have resulted in the lower NDFD, as sorghum grain provided more available starch to compete with fiber degradation which may have resulted in depression of fiber digestion (Van Soet, 1989). In addition, the finer feed ingredient particle size used in pellets can result in a faster passage of the feed from the rumen and loss of potentially digestible fiber which may depress the overall digestibility of cell walls in the animals nutrition (Van Soet, 1989).

An increase in feed intake can result in the depression of nutrient digestibility as a result of an often associated increase in the passage rate of digesta. In this trial, the B. amyloliquefaciens H57 supplement improved the rumen environment and rumen fermentation in a manner such that the higher feed intake induced by B. amyloliquefaciens H57 was not accompanied by a depression in digestibility. Rumen pH in the H57 group was higher than in the control group, 7.11 versus 6.78, respectively. Higher pH may relate to the lower total fatty acid concentration which was 39.2 mmol/1 for B. amyloliquefaciens H57 and 65.1 mmol/1 for the control or the contamination of saliva. The total VFAs are influenced by several factors such as amount of water animals consume, sampling and the absorption rate of VAFs and ammonium in the rumen. The ruminal ammonia concentration in the B. amyloliquefaciens H57 group was 69.1 mg/1, lower than the 111.1 mg/l in the control group. The concentration of ammonia in the Treatment group was still in the optimal range of 60-80 mg/l (Freer, 2007)) for the activity of rumen flora. Lower ruminal ammonia concentration in the B. amyloliquefaciens H57 group may indicate less protein degradation in the rumen, resulting in an increase of by-pass protein to the abomasum where it can be digested by the animal.

Supplementing the diet of ewes in late pregnancy with B. amyloliquefaciens H57 improved their nitrogen balance. Similar results from supplementation of feed with Bacillus probiotics has been reported to improve nitrogen retention in birds (Mohan et al., 1996) and fish (Faramarzi et al., 2012). Nitrogen retention was much higher in the Treatment than in the Control, 6.47 g/d compared to 3.03 g/d. The combination of an increased in nitrogen intake and a decrease in urinary nitrogen excretion may resulted in higher nitrogen retention in Treatment group, but the difference both in nitrogen intake or urinary nitrogen excretion between two groups was not significant (P>0.05).

In conclusion, Bacillus amyloliquefaciens strain H57 used as a probiotic improved feed intake, liveweight gain, nitrogen retention in pregnancy, and performance in early lactation of first-parity ewes.

### Example 5

### The Probiotic Performance of Bacillus amyloliquefaciens Strain H57 in Dairy Calves

### Microbial Profiling of the Rumen

At week 4 of age, calves in a same age group were assigned into the two treatment groups, Control and H57 according to their initial weight. The H57 treatment group calves were given free ad libitum access to starter pellets containing 10⁹ cfu H57/kg DM, as fed. In the test period, calves were fed 61 per day of whole milk, twice daily and ad libitum pellets. When calf liveweight was about 70 kg and they were eating 700 g/day pellets for 3 consecutive days, afternoon milk was withdrawn for 3 days and then all milk. After weaning, calves continued to be fed ad libitum pellets until 12 weeks old as per Example 7. Rumen fluid samples were then collected from the 24 dairy calves (12× control, 12× treatment) using a stomach tube and processed as per Example 4 above.

### Rumen Microbial Changes of Dairy Calves

Analysis of the rumen microbial community in dairy calves fed the probiotic B. amyloliquefaciens H57 demonstrated no substantial differences in bacterial population and a fairly similar bacterial population of all animals was observed within both treatment groups (data not shown).

### Example 6

To determine which lipopeptides are produced by Bacillus amyloliquefaciens H57, samples of culture medium were analysed by matrix-assisted laser desorption ionization time-of-flight mass spectrometry (MALDI-TOF MS). Briefly, the culture medium was diluted 1:100 with ultrapure water and the diluted sample (1 uL) was mixed with a saturated solution of α-cyano-4-hydroxycinnamic acid in 0.1% (v/v) trifluoroacetic acid, 70% (v/v) aqueous acetonitrile (1 uL) and allowed to dry in situ prior to analysis. Mass spectrometry showed peaks corresponding to the expected molecular weights for surfactin (C13-C16; m/z (M+Na⁺) 1044, 1058; (M+K⁺) 1046, 1060, 1074, 1088), fengycin-A (C15:0-C17:0; m/z (M+H⁺) 1450, 1464, 1478; (M+Na⁺) 1486; (M+K⁺) 1502) and fengycin-B (C15:0-C17:0; m/z (M+H⁺) 1492; (M+K⁺) 1516, 1530, 1544).

### Example 7

### Effect of Probiotic Bacillus amyloliquefaciens on Growth Performance and Diarrhoea in Dairy Calves

### Methodology

Twenty four calves (weight: 51.4±5.7 kg; age: 28±3 days) were selected from the University of Queensland Gatton dairy farm for the trial. The farm procedure for new-born calf management was followed before assignment into the trial which, included ingesting colostrum for the first two days in individual pens. The calves were then moved to group pens and fed up to 10 L per day and ad libitum antibiotic free starter pellets by robotic feeder. Due to the variation in birth date, calves were blocked into 3 groups on a weekly basis according to birth date and sex to enable a common starting date for starting the H57 supplement. At week 4 of age, calves in a same age group were assigned into the two treatment groups, Control and H57 according to their initial weight. The H57 treatment were given free ad libitum access to starter pellets containing 10⁹ cfu H57/kg DM, as fed.

The trial included 2 periods: the test period from week 4 to week 12 and the carry over period from week 13 to 19 of age. In the test period, calves were fed 61 per day of whole milk, twice daily and ad libitum pellets. When calf liveweight was about 70 kg and they were eating 700 g/day pellets for 3 consecutive days, afternoon milk was withdrawn for 3 days and then all milk. After weaning, calves continued to be fed ad libitum pellets until 12 weeks old. The weaning age was marked on the day that milk was withdrawn from the diet. Calves were kept in individual pens with a concrete floor covered with straw and a solid plastic panel between pens (2.2 m length×1.6 m width×1.2 m length) to prevent contact between calves. Fresh straw was added daily in the morning and changed twice weekly. Pellet intake was recorded weekly. An amount of pellets estimated to cover the week's use was weighed into a separate bin for each calf from which fresh pellets were supplied daily to each calf at 7 am after removing the residues from the previous days feed. Residues were stored and weighed weekly. Milk intake was measured daily, milk refusal was collected and measured after 30 minutes of feeding. Milk samples were analyzed fortnightly for milk composition. In the carry over period, all calves were kept in the same paddock supplying grass grazing and provided an ad libitum supplement control pellets without H57 and mix hay of oaten and lucerne (60:40). In this example the data recording and analysis is based on calf age in weeks.

Calves were checked twice daily for any abnormal signs such as lost appetite, scours (calf diarrhoea), infections in joints or navel, respiratory problems (nasal discharge, cough). All calves were checked weekly by a veterinarian to assess their health. Calves were treated for scours and respiratory illness when they had raised temperature, lethargy, were off their feed or had scours for longer than 2 days. Where required, Electrolytes (Vytrate® Duo Sachets, Jurox Pty Ltd, NSW 2320 Australia) and long action Oxytetracycline and Ketoprofen were administered according to manufacturer's recommendations.

All experimental procedures were approved by The University of Queensland Animal Ethics Committee.

The ingredients of starter pellets included (g/kg DM) wheat grain: 113, Sorghum grain: 558, Canola meal: 170, Soybean meal: 136, Legume hulls: 90, Molasses: 34, Limestone: 17, Nitrate salt: 6, Calcium Chloride: 6, Premix: 2 (Premix (mg/kg, unless stated): Vitamin A, 3000 IU/g; Vitamin D3, 250 IU/g; Vitamin E, 2500; Ion, 7500; Zinc, 25000; Manganese, 1000; Selenium, 50; Molybdenum, 500; Cobalt, 500; Iodine. 500). The chemical composition of the experimental diet is displayed in Table 24.

The pelleted feed was prepared at the Ridley Toowoomba plant with control feed prepared first. H57 inoculum was prepared in a 100 L fermenter at the University of Queensland, the bacteria separated out in a Sharples industrial centrifuge, the pellet resuspended in bentonite, frozen at −20° C. and freeze dried. The material was then ground to a powder and mixed progressively with 200 Kg of sorghum ground finely to pass a 1 mm sieve. The bentonite inoculum added contained 10¹³ spores and this resulted in 10⁶ spores/gram of pelleted feed in the two tonne mix, which was then stored in 25 kg plastic bags. This H57 population level remained during the duration of the trial. Bagged feed was stored at 12° C. and enough feed removed to ambient conditions (20 to 38° C.) for each weeks feeding.

TABLE 24
The chemical compositions of experimental diet for calves

| Compositions (% DM) | Control Pellets | H57 Pellets | Milk |
|---|---|---|---|
| Metabolic energy (ME) (MJ/kg) | 14.0 | 13.9 | 21.99 |
| Dry matter (DM) (%) | 89.3 | 89.1 | 13.5 |
| Crude protein (CP) | 19.2 | 19.0 | 3.15 |
| Fat | 5.65 | 5.75 | 3.93 |
| Neutral detergent fiber (NDF) | 10.7 | 11.3 | NA |
| Acid detergent fiber (ADF) | 7.40 | 7.85 | NA |
| Total digestible nutrients (TDN) | 85.3 | 85.0 | NA |
| Starch | 42.4 | 43.0 | NA |
| Lignin | 2.00 | 1.90 | NA |
| Ash | 6.85 | 7.00 | 0.8 |
| Potassium (K) | 0.78 | 0.78 | NA |
| Chloride (Cl) | 0.75 | 0.76 | NA |
| Calcium (Ca) | 1.01 | 1.10 | NA |
| Magnesium (Mg) | 0.20 | 0.21 | NA |
| Phosphorus (P) | 0.41 | 0.41 | NA |
| Sulfur (S) | 0.27 | 0.27 | NA |
| Sodium (Na) | 0.19 | 0.19 | NA |
| Iron (Fe) | 165 | 177 | NA |
| Zinc (Zn) | 105 | 111 | NA |
| Copper (Cu) | 14.5 | 13.8 | NA |
| Molybdenum (Mo) | 1.93 | 2.28 | NA |

NA: not available

Samples of pellets were collected weekly and stored at −20° C. At the end of the test period, weekly pellet samples were combined and two subsamples from each group were taken for analysis of the compositions (Table 24) by Dairy One Forage Laboratory (730 Warren Road, Ithaca, N.Y. 14850).

Blood samples were collected at week 4, week 12 and week 19 of age, 5 hours after morning feeding. The blood samples were centrifuged at 3500 rpm for 10 minutes at 4° C. (Beckman J6-MI) within 30 minutes of collection to separate the plasma and blood cells. Plasma was analyzed for metabolytes including aspartate aminotransferase (AST), glutamate dehydrogenase (GLDH), gamma glutamyl transferrase (GGT), total bilirubin (TBIL), cholesterol (CHOL), creatine phosphokinase (CPK), creatinine, urea; electrolytes, and non-esterified fatty acids (NEFA) using an Olympus AU400 auto-analyzer (Beckman Coulter Diagnostic Systems Division; Melville, N.Y.C, USA), following manufacturers' procedures.

Rumen samples were collected at the start and end of each period of calf management, at 4 hours after morning feeding using an oesophageal catheter. Rumen pH was measured immediately after collection, then two sub-samples of rumen fluid were collected, 4 ml (+1 ml of 20% metaphosphoric acid) for volatile fatty acids (VFAs) analysis, and 8 ml (+20% sulphuric acid) for ammonia (NH₃) analysis.

### Statistical Analysis

Analyses of feed intake and liveweight change were conducted using a GLM in STATISTICA 8. Sum of squares were partitioned into effects for treatment and time along with possible interactions. The initial liveweight of the calves was used as the covariate, calf within treatment was included as a random effect and time was considered as a repeated factor. Rumen characteristics and plasma parameters were analyzed using one-way ANOVA in STATISTICA 8. The model includes the fixed effect of treatment, the random effect of calf within treatment and random residual error.

### Results

### Liveweight and Daily Weight Gain.

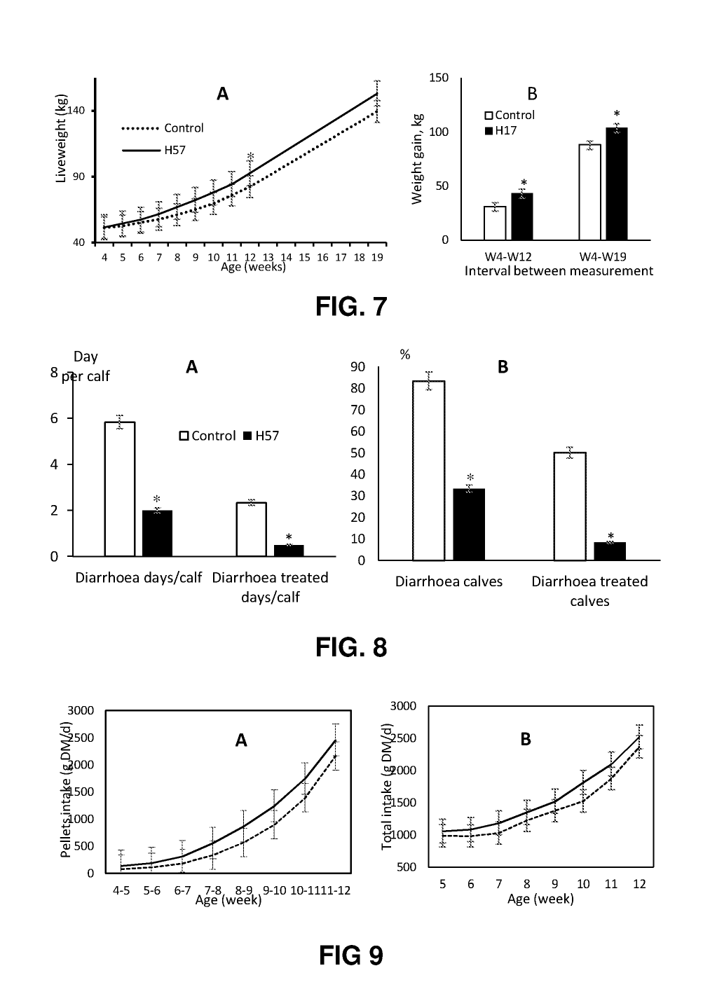

The initial liveweight at week 4 of age was identical between two groups (Table 25 and FIG. 7). H57 improved daily weight gain (DWG) by 36%. The H57 calves gained 12.4 kg more than that of the Control calves. At the end of test period, liveweight of the H57 calves was 11% higher than the Control calves. The DWG of H57 calves during carry the over period was higher than the Control calves (P=0.06) and the final liveweight of H57 calves was 20% higher than the Control calves (P<0.01).

TABLE 25
Effect of Bacillus amyloliquefaciens
on growth performance of dairy calves

| | Control | H57 | |
|---|---|---|---|
| | Mean ± s.e | Mean ± s.e | P |
| Liveweight (week 4), kg | 51.1 ± 1.44 | 51.2 ± 1.51 | 0.92 |
| Liveweight (week 12), kg | 82.3 ± 3.28 | 94.8 ± 3.44 | 0.02 |
| Liveweight (week 19), kg | 139.4 ± 4.56 | 155.3 ± 4.79 | 0.03 |
| Daily weight gain | 551 ± 52.4 | 767 ± 55.3 | 0.01 |
| (week 4-12), g/d | | | |
| Daily weight gain | 866 ± 126 | 1232 ± 133 | 0.06 |
| (week 12-19), g/d | | | |
| Milk intake, g DM/d | 596 ± 36.3 | 521 ± 38.1 | 0.17 |
| Pellet intake, g DM/d | 740 ± 91.7 | 1001 ± 96.8 | 0.07 |
| Total intake, g DM/d | 1309 ± 83.4 | 1526 ± 87.5 | 0.09 |
| Feed efficiency (feed: gain) | 2.90 ± 0.10 | 2.46 ± 0.11 | <0.01 |
| Days to wean | 70.6 ± 2.19 | 61.9 ± 2.31 | 0.02 |

DM: dry matter;
s.e: standard error;
g/d: gam/day

### Diarrhoea

Diarrhoea occurred in both groups mainly during the pre-weaned period when calves were fed milk and pellets. During test period, H57 reduced diarrhoea occurrence by 40% and the need to treat calves by 25% (P<0.05, FIG. 8B). The duration of diarrhoea calculated as the number of days per each calf averaged over all incidences, was 3.5 days longer in the Control calves than in the H57 calves (P<0.05). The duration of diarrhoea treatment required for H57 calves was one third of that in Control calves (P<0.05, FIG. 8A). The veterinarian managing the calf health did not know which calves belonged to which treatment.

One calf in each group developed a respiratory problem, while the H57 calf was able to recover, the control calf had to be treated with antibiotic therapy for 3 days.

### Days to Wean

H57 advanced the weaning age by 1 week cf. the Control calves (P<0.05, Table 25). At the end of the test period at week 12 of age, all the H57 calves were weaned, while two (17%) Control calves did not meet the weaning criteria and were then weaned abruptly on that day.

Daily milk intake was the same for H57 and control calves up to weaning but the pellet intake tended to be higher for the H57 calves (P=0.07, Table 25, FIG. 9). Total Dry Matter Intake (DMI) of the H57 calves was 12.1% higher than the Control calves (P=0.09). The feed efficiency (FE) calculated as kilogram DMI per kilogram of weight gain was improved by 14% by the H57 (P<0.05). The H57 calves consumed about 0.45 kg less DMI for a kilogram of weight gain than the control calves (P<0.05)

### Rumen Characteristics

In the test period, H57 did not influence ruminal pH, ammonia and total VFAs, but increased molar ratio of valeric acid as a percentage of total VFA's by 34% (P<0.05, Table 26) and potentially increased molar Butyric (P=0.08). No differences in rumen characteristics were found between the two treatments at the end of the carry over period.

TABLE 26
Effect of Bacillus amyloliquefaciens H57 on rumen characteristics of dairy calves

| | | Week 12 | | | Week 19 | | |
|---|---|---|---|---|---|---|---|
| | Week 4 | Control | H57 | P | Control | H57 | P |
| pH | 5.76^{a} ± 0.14 | 5.61^{a} ± 0.17 | 5.36^{a} ± 0.08 | 0.10 | 6.28^{b} ± 0.13 | 6.51^{b} ± 0.15 | 0.33 |
| Rumen NH₃, | 291.7^{a} ± 25.7 | 74.4^{b} ± 14.2 | 82.3^{b} ± 15.7 | 0.85 | 120.3^{c} ± 11.6 | 102.3^{c} ± 9.87 | 0.49 |
| mg/L | | | | | | | |
| Total VFAs, | 52.1 ± 3.99 | 108.4 ± 11.7 | 119.0 ± 11.5 | 0.44 | 87.3 ± 7.62 | 81.8 ± 6.55 | 0.58 |
| mmol/L | | | | | | | |
| Acetic, % | 23.1 ± 1.45 | 48.1 ± 5.29 | 46.8 ± 5.03 | 0.51 | 52.3 ± 2.72 | 51.7 ± 2.53 | 0.51 |
| Propionic, % | 16.1 ± 1.86 | 39.9 ± 5.94 | 38.6 ± 5.37 | 0.44 | 33.9 ± 3.75 | 33.5 ± 3.50 | 0.32 |
| Iso-butyric, % | 0.75 ± 0.09 | 1.49 ± 0.08 | 1.29 ± 0.07 | 0.39 | 2.08 ± 0.07 | 2.21 ± 0.06 | 0.65 |
| Butyric, % | 7.52 ± 0.83 | 7.39 ± 0.82 | 8.61 ± 1.39 | 0.08 | 8.21 ± 1.27 | 8.58 ± 0.88 | 0.82 |
| Iso-valeric, % | 1.35 ± 0.15 | 0.59 ± 0.12 | 0.59 ± 0.09 | 0.83 | 0.76 ± 0.09 | 0.88 ± 0.08 | 0.89 |
| Valeric, % | 3.27 ± 0.47 | 2.47 ± 0.33 | 4.10 ± 0.98 | 0.03 | 2.66 ± 0.45 | 3.04 ± 0.35 | 0.55 |
| A/P ratio | 1.72 ± 0.18 | 1.44 ± 0.25 | 1.27 ± 0.08 | 0.52 | 1.80 ± 0.17 | 1.76 ± 0.22 | 0.38 |

### Plasma Parameters

TABLE 27
Effect of Bacillus amyloliquefaciens H57 on plasma biochemistry of calves

| | | Week 12 | | | Week 19 | | |
|---|---|---|---|---|---|---|---|
| | Week 4 | Control | H57 | P | Control | H57 | P |
| CPK, U/L | 166.2 ± 11.6 | 122.3 ± 9.50 | 99.3 ± 14.1 | 0.11 | 151.2 ± 8.75 | 154.0 ± 11.3 | 0.98 |
| AST, U/L | 38.7 ± 2.63 | 53.7 ± 5.89 | 46.7 ± 4.49 | 0.30 | 63.1 ± 2.84 | 63.4 ± 2.48 | 0.99 |
| GGT, U/L | 48.8 ± 7.05 | 20.3 ± 2.33 | 16.0 ± 1.01 | 0.06 | 14.6 ± 0.40 | 13.3 ± 0.39 | 0.01 |
| ALP, U/L | 147 ± 17.3 | 233 ± 31.8 | 238 ± 34.4 | 0.74 | 217 ± 16.2 | 237 ± 28.1 | 0.61 |
| GLDH, U/L | 31.7 ± 5.01 | 47.0 ± 9.50 | 32.3 ± 5.57 | 0.15 | 38.3 ± 13.1 | 27.2 ± 2.85 | 0.49 |
| Glucose, mmol/L | 5.02 ± 0.17 | 5.51 ± 0.26 | 5.32 ± 0.23 | 0.65 | 5.46 ± 0.16 | 5.70 ± 0.14 | 0.32 |
| NEFA, mmol/L | 0.22 ± 0.02 | 0.09 ± 0.01 | 0.08 ± 0.01 | 0.79 | 0.12 ± 0.03 | 0.14 ± 0.04 | 0.65 |
| Triglyceride, mmol/L | 0.36 ± 0.06 | 0.44 ± 0.04 | 0.30 ± 0.03 | 0.01 | 0.26 ± 0.02 | 0.23 ± 0.02 | 0.29 |
| BHB, mmol/L | 0.12 ± 0.01 | 0.24 ± 0.03 | 0.29 ± 0.03 | 0.12 | 0.26 ± 0.02 | 0.20 ± 0.01 | 0.03 |
| Cholesterol, mmol/L | 2.27 ± 0.21 | 1.77 ± 0.20 | 1.72 ± 0.15 | 0.85 | 1.33 ± 0.10 | 1.62 ± 0.10 | 0.05 |
| Urea, mmol/L | 3.69 ± 0.21 | 2.73 ± 0.17 | 2.48 ± 0.13 | 0.33 | 4.28 ± 0.23 | 4.20 ± 0.29 | 0.87 |
| Creatinine, μmol/L | 81.0 ± 2.54 | 65.5 ± 3.90 | 62.9 ± 2.45 | 0.54 | 62.2 ± 1.91 | 62.6 ± 1.82 | 0.85 |
| Globulin, g/L | 25.6 ± 1.30 | 29.9 ± 2.16 | 24.9 ± 2.14 | 0.03 | 30.2 ± 0.53 | 32.9 ± 3.03 | 0.35 |
| Albumin, g/L | 27.0 ± 0.82 | 29.3 ± 2.47 | 28.2 ± 1.92 | 0.75 | 30.7 ± 0.48 | 31.8 ± 0.76 | 0.28 |
| Total Protein | 52.6 ± 1.84 | 59.3 ± 4.21 | 53.3 ± 3.81 | 0.23 | 60.9 ± 0.55 | 64.7 ± 2.74 | 0.18 |
| Total Bili, μmol/L | 3.54 ± 0.18 | 3.28 ± 0.29 | 3.03 ± 0.25 | 0.42 | 3.00 ± 0.12 | 2.91 ± 0.19 | 0.68 |
| Sodium, mmol/L | 134.4 ± 0.99 | 139.5 ± 6.03 | 133 ± 4.26 | 0.37 | 130.2 ± 1.66 | 130.6 ± 1.44 | 0.88 |
| Calcium, mmol/L | 2.14 ± 0.11 | 2.64 ± 0.15 | 2.37 ± 0.12 | 0.12 | 2.38 ± 0.03 | 2.36 ± 0.04 | 0.89 |
| Magnesium, mmol/L | 0.64 ± 0.03 | 0.81 ± 0.06 | 0.79 ± 0.04 | 0.77 | 0.79 ± 0.02 | 0.80 ± 0.02 | 0.92 |
| Potasium, mmol/L | 5.10 ± 0.08 | 4.67 ± 0.12 | 4.41 ± 0.16 | 0.23 | 4.08 ± 0.05 | 4.08 ± 0.09 | 0.97 |
| Chloride, mmol/L | 99.9 ± 0.59 | 104.1 ± 3.49 | 99.6 ± 2.74 | 0.19 | 96.1 ± 1.14 | 96.2 ± 1.08 | 0.91 |
| Phosphate, mmol/L | 2.32 ± 0.06 | 2.66 ± 0.22 | 2.63 ± 0.23 | 0.97 | 2.47 ± 0.10 | 2.41 ± 0.09 | 0.68 |
| Bicarbonate, mmol/L | 26.6 ± 0.47 | 27.8 ± 1.34 | 26.6 ± 0.84 | 0.48 | 25.4 ± 0.60 | 25.3 ± 0.72 | 0.91 |

NEFA: Non-esterified fatty acids,
CPK: Creatinine Kinase,
AST: Aspartate aminotransferase,
ALP: Alkaline phosphatase,
GLDH: Glutamate dehydrogenase,
GGT: Gamma-glutamyl Transferase,
BHB: B-hydroxybutyrate

At week 12 of age, plasma GGT, globulin and triglycerides were higher in the Control than in the H57 group (P<0.05, Table 27). At week 19 of age, plasma BHB and GGT were higher in the Control than in the H57 group. Cholesterol was higher in H57 calves than in the Control.

### DISCUSSION

The current study showed that starter pellets containing the probiotic H57 improved growth performance and reduced the occurrence of diarrhoea in young dairy calves. Diarrhoea is one of the most common health problems contributing to the mortality in young ruminants. The high level of diarrhoea present in the current study for control calves may be associated with the antibiotic free pellets and the hot temperatures on some days where it reached 40.5° C. in the calf shed for a period. However, the H57 reduced not only the percentage of calves which had diarrhoea but also the duration of diarrhoea. H57 calves not only grew faster but were also healthier. While Control calves spent a lot of time lying down in the pens, H57 calves spent more time standing and looking for feed. H57 calves also drank milk from buckets much quicker than control calves. More calves developed diarrhoea and for longer in Control treatment than for H57 calves and took longer to cure.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, gene and protein sequences identified by accession number, patent and scientific literature referred to herein is incorporated herein by reference in their entirety.

1. A probiotic composition comprising, consists of or consists essentially of a microbial culture of Bacillus amyloliquefaciens strain H57 bacteria and an acceptable carrier.

2. The probiotic composition of claim 1, further comprising a probiotic microorganism of one or more genera selected from the group consisting of Lactobacillus, Bifidobacterium, Enterococcus, Streptococcus, Bacillus, Propionibacterium, Enterococcus, Streptococcus, Pediococcus, Clostridium, Aspergillus, Candida, Saccharomyces, Megasphaera and any combination thereof.

3. The probiotic composition of claim 1, wherein the microbial culture comprises, consists or consists essentially of spores of Bacillus amyloliquefaciens strain H57 bacteria.

4. The probiotic composition of claim 1, wherein the microbial culture is lyophilised and/or freeze dried.

5. The probiotic composition of claim 1, wherein the probiotic composition is formulated as an animal feed composition.

6. The probiotic composition of claim 5, which comprises a pelleted, granular and/or particulate feed material.

7. The probiotic composition of claim 6, wherein the feed material is selected from the group consisting of palm kernel meal, wheat, sorghum, corn, soybean meal and any combination thereof.

8. The probiotic composition of claim 5, wherein the animal feed composition is or comprises a lick block.

9. The probiotic composition of claim 1, wherein the microbial culture is present at a concentration of about 1×10⁶ to about 1×10¹⁰ CFU per gram of the composition.

10. The probiotic composition of claim 1, wherein the microbial culture is present at a concentration so as to provide a dose of about 1×10⁷ to about 1×10¹¹ CFU per day to an animal fed the probiotic composition.

11. The probiotic composition of claim 1, substantially free of antibiotics and/or antimicrobial agents.

12. The probiotic composition of claim 1, which is steam pelleted.

13. A method of preventing and/or treating a disease, disorder or condition in an animal, wherein said disease, disorder or condition is responsive to a probiotic, including the step of administering to said animal a therapeutically effective amount of a probiotic composition comprising a microbial culture of Bacillus amyloliquefaciens H57 bacteria to thereby prevent and/or treat the disease, disorder or condition.

14. The method of claim 13, wherein the disease, disorder or condition is diarrhoea.

15. A method for improving or increasing feed conversion efficiency, dietary intake, weight gain, egg production and/or egg quality in a monogastric animal including the step of administering a probiotic composition comprising Bacillus amyloliquefaciens H57 bacteria to the monogastric animal in an amount effective to facilitate improving or increasing feed conversion efficiency, dietary intake, weight gain, egg production and/or egg quality in the monogastric animal.

16. The method of claim 15, wherein once administered the Bacillus amyloliquefaciens H57 bacteria strain bacteria colonizes, at least temporarily, at least a portion of a gastrointestinal tract of the monogastric animal.

17. The method of claim 15, wherein administration of the probiotic composition modulates one or more species or genera of microbial flora in at least a portion of a gastrointestinal tract of the monogastric animal.

18. The method of claim 15, wherein the probiotic composition is administered by mixing the composition with a feed material and/or spraying the composition onto a feed material prior to feeding.

19. The method of claim 17, wherein the probiotic composition is administered by adding the probiotic composition to the monogastric animal's drinking water prior to feeding.

20. A method for modulating microbial flora in at least a portion of a gastrointestinal tract of an animal including the step of administering a probiotic composition comprising Bacillus amyloliquefaciens H57 bacteria bacteria to the animal in an amount effective to achieve said modulation.

21. The method of claim 20, wherein the microbial flora include one or more bacteria of a genus selected from the group consisting of Acidaminococcus, Akkermansia, Anaerovibrio, Arthromitus, Bacteroides, Blautia, Butyrivibrio, Faecalibacterium, Coprococcus, Lachnobacterium, Lachnospira, Lactobacillus, Megasphaera, Methanobrevibacter, Mitsuokella, Prevotella, Pseudoramibacter, Roseburia, Ruminobacter, Ruminococcus, Selenomonas, Shuttleworthia, Sphaerochaeta, Staphylococcus, Streptococcus, Succiniclasticum, Succinivibrio, Turicibacter and any combination thereof.

22-23. (canceled)

24. A method for manufacturing a probiotic composition including the steps:
(i) growing a microbial culture of Bacillus amyloliquefaciens H57 bacteria bacteria in a suitable media;
(ii) substantially isolating the microbial culture from the media;
(iii) inducing sporulation of the microbial culture before and/or after step (ii); and
(iv) combining spores of Bacillus amyloliquefaciens H57 bacteria with an acceptable carrier.

25. The method of claim 24, further including the step of lyophilising and/or freeze drying the spores after steps (iii) and/or (iv).

26-27. (canceled)

28. The method of claim 13, wherein once administered the Bacillus amyloliquefaciens H57 bacteria strain bacteria colonizes, at least temporarily, at least a portion of a gastrointestinal tract of the monogastric animal.

29. The method of claim 13, wherein administration of the probiotic composition modulates one or more species or genera of microbial flora in at least a portion of a gastrointestinal tract of the monogastric animal.

30. The method of claim 13, wherein the probiotic composition is administered by mixing the composition with a feed material and/or spraying the composition onto a feed material prior to feeding.

31. The method of claim 13, wherein the probiotic composition is administered by adding the probiotic composition to the monogastric animal's drinking water prior to feeding.
